SEQUENCE LISTING (213 sequences)

SEQ ID NO: 1 - DNA, 21 bp, Artificial sequence
  PCR primer
cgatttatcaggcactcatcg

SEQ ID NO: 2 - DNA, 20 bp, Artificial sequence
  PCR primer
caaataggcctccatcacca

SEQ ID NO: 3 - DNA, 18 bp, Artificial sequence
  PCR primer
atgaacggagcgcatcac

SEQ ID NO: 4 - DNA, 18 bp, Artificial sequence
  PCR primer
cgaccgaagcttgggaat

SEQ ID NO: 5 - DNA, 1317 bp, Mycobacterium avium
  CDS (57)..(1292)
tcgcgtgaggctctgtggtgaaacgaccaaggatcactaccgagaggaacatcgcgatggccctggatcagtctgccttgctggaggtgctggacgcattacgcaatgccgatgccgctgatcggatcaagcaggccgccgagacgatttatcaggcactcatcgatgcggagctgacggcggtgatcggcgccggtccgcatgaacggagcgcatcacgaaccaaccagcgcaatgggtctcggccgcgcacactgtcgacgatcgctggcgatttggagttgcggattcccaagcttcggtcgggctcgttcttcccggcgctgctggagcggcgccgccgggtcgatcagtgcttgttcgcggtggtgatggaggcctatttgcacggcacctccacccgcaaagtcgacgatttggttaaagccctcggcgcggacgccggcatctccaaaagcgaggtctcccggatttgcgccgatctggacaccgaggtaggcgcctttcgggaccggcctctatccgagcagcacttcccgtatgtgttcctcgacgccacttactgcaaggctcgggtcaaccatcgggtggtctcccaagcggtcgtcatcgccaccggtgtcgcggttgacggacgccgtgaagtgctcggcttcgacgtcggtgactccgaggacggcgcgttttggacggcgtttctgcgctcactgaaaacccgcggcctgtccggggtgcagctggtgatctctgatgcccacaccgggttgcgcagcgccatcgaggccatcctcatcggcgcatcttggcaacgatgccgggtgcacttcctgcgcaacgtgctcgcccaggtgcccaagggatcagccgagatggtcgccgcagcgatccgcaccatcttcgcccaacccgacgccgaacatgtgcgagagcaactcgacaccatcgccggcatgctcggccgccaactgcccaaggtcgaaacgatgctgcgcgaggccgccgacgacatcaccgcattcgccgatttccctgtcctgcactggaaaaagatctggagcaccaaccccctcgaacgactcaacaaggagatcaaacgccgcaccgacgtcgtcggcgtcttccccaaccccgccgcgctgctacggctggccggctccgttctcgtcgaagcccacgacgaatggcaagtcgccgaaaagcgctatctctccgagaccaccctggccctattacatccccgctccgattcagccgaccaatccgttgccgtccccgccgccatcacggcatagtggaaactcgcagagcctcaggcga

SEQ ID NO: 6 - protein, 411 aa, Mycobacterium avium
MALDQSALLEVLDALRNADAADRIKQAAETIYQALIDAELTAVIGAGPHERSASRTNQRNGSRPRTLSTIAGDLELRIPKLRSGSFFPALLERRRRVDQCLFAVVMEAYLHGTSTRKVDDLVKALGADAGISKSEVSRICADLDTEVGAFRDRPLSEQHFPYVFLDATYCKARVNHRVVSQAVVIATGVAVDGRREVLGFDVGDSEDGAFWTAFLRSLKTRGLSGVQLVISDAHTGLRSAIEAILIGASWQRCRVHFLRNVLAQVPKGSAEMVAAAIRTIFAQPDAEHVREQLDTIAGMLGRQLPKVETMLREAADDITAFADFPVLHWKKIWSTNPLERLNKEIKRRTDVVGVFPNPAALLRLAGSVLVEAHDEWQVAEKRYLSETTLALLHPRSDSADQSVAVPAAITA

SEQ ID NO: 7 - DNA, 2263 bp, Mycobacterium avium
  misc_feature (1)..(2263): Mycobacterium avium subspecies paratuberculosis insertion sequence ISMav2
gagctcggccagcacttcatccgggtgaattccgtgcaccccaccaacgtgaacacaccgatgttcatgaacgaggggacgatgaggctgttccggccggacctgaagaaccccggcccggacgacctgaaggtcgccgcgcagttcatgcatgtgctgccggtcggctgggtggagccggtggacatcagcaacgccgtgctgttcctggcctccgacgaatcgcgttacatcacaggtcttccggtcaccctcgacgccggcagcatgctcaagtagccgcgccggcatctcgctcaccggcggtgaggatcatcccggtggccaggggcgggatcgaaccgccgaccttccgcttttcaggcggacgctcgtaccgactgagctacctggccggaaggcagcgagtggctcgccgcgctggcgaccctgacgggactcgaacccgcgacctccgccgtgacagggcggcgcgctaaccaactgcgccacagggccttgctgctgctccgcgtcgccgcgttgcgtaccccctacgggattcgaacccgcgctaccgccttgaaagggcggcgtcctaggccgctagacgaagggggccagaaccgaatctctccggggtactcgcaacgtggtttcgttgggagccacgtcagcttaggtcaccgtgggcccaatcctcaaacgagcccggttttgggtccaagtatcctgaaactccgcggcccctatagctcagttggtagagctacggacttttaatccgcaggtcctaggttcgagtcctagtgggggcaccagatgtatgtatgacgtcggctgatgtgtgacgttttgtcttcggttgatgtgtgacggtgtttcggttgatgcgtgacagttgtttcggctgatcggtgacactccctagatgagggagttaagtgtggctgagcagcggtatcaggccgtgatggcggtaatcagtgacgggttgtcggtgtcgcaggccgcagagaagttcggggtggcgcgtcagacgctgcaccgatggctggcccggtatgaagccgcgggcctgaaggggctggtggatcggtcgcatcggccggtgagttgtccgcatcagatgctggcggtagtggaggcggcggtgttggagttgcgccgctcgcggccctattgggggccgcggcggttggtgttcgagttggccaagcgaggtgtccatccggtgccgtcggagtcgccggtgtatcgagcgctggtgcgggccggtctgatcgaccccgcgatgcgagatcgacggtcgcgcaaatggaagcgctgggagcgcggggcgccgatggagttgtggcagctcgacatcgtcggcgggttcccgctggccgatgggaccagcgccaaagccctgaccggcatcgatgatcattcccggatgtgtgtgtgcgccaagctgatggcccgtgagcgcacccgcgcggtctgcgacggattacgggcggcgctggccgcttacggggtgcctgagcagatcttgaccgataacggcaaggtgttcaccgagcggttttgtcatccaccggtcgaggtgctctttgatgcgatctgccgcgagcacggcatcgaacatctgttgacccagccgcgcagcccgaccacgaccggcaaaatcgagcagtttcaccgcagtctgcgcgctgagttccttagcggccgtgagcctttcaccaacctcaaggtcgctcagcaggcgctcgatgagtgggtcgaggactacaacaccacccggccgcaccaagccctaaagatgatcacaccggctcaacggtttcacgccggtgcgccggcatcaccaccgtcgaactcgtgtgcccgacacgtcgaccgcagtggtgatgactgggtgagtcggcgggtgtgctccaacggcatcgtgtgcgtgtcctggcagcaggtctgtatcgggcgccactatgccggcgcccgctgcgatgtccacgtcgatggggacctgctgaggttttgggtcggcgacaatctggtcaaaaccgccgcgcgcaccagccgcggcgaggtacgaaacaaacaggccctgcgcaccaacgcaccggcctaaaacacaacccagagtgtcaccgatcaaccgacatagaaatgtcaccgagcaaccgaccctgaacaggggcaccagagagcagccccgcccgctcaggtggtcgacgatgtggctgcgctcgccgtggtagaccaggtcattgaaatcgacctcgaacccgagctc

SEQ ID NO: 8 - DNA, 20 bp, Mycobacterium avium
  misc_feature (1)..(20): Primer F1
gtcattcagaatcgctgcaa

SEQ ID NO: 9 - DNA, 20 bp, Mycobacterium avium
  misc_feature (1)..(20): Primer F2
tggcgtcagctattggtgta

SEQ ID NO: 10 - DNA, 20 bp, Mycobacterium avium
  misc_feature (1)..(20): Probe F1F2
aactcgaacacacctgggac

SEQ ID NO: 11 - DNA, 20 bp, Mycobacterium avium
  misc_feature (1)..(20): Primer F3
tcctctccttcgtcaccaac

SEQ ID NO: 12 - DNA, 20 bp, Mycobacterium avium
  misc_feature (1)..(20): Primer F4
atgaaatgggcgtctaccag

SEQ ID NO: 13 - DNA, 20 bp, Mycobacterium avium
  misc_feature (1)..(20): Probe F3F4
gtcattcagaatcgctgcaa

SEQ ID NO: 14 - DNA, 20 bp, Mycobacterium avium
  misc_feature (1)..(20): Primer F5
gtcattcagaatcgctgcaa

SEQ ID NO: 15 - DNA, 20 bp, Mycobacterium avium
  misc_feature (1)..(20): Primer F6
cgtcagctattggtgtaccg

SEQ ID NO: 16 - DNA, 20 bp, Mycobacterium avium
  misc_feature (1)..(20): Probe F5F6
aactcgaacacacctgggac

SEQ ID NO: 17 - DNA, 20 bp, Mycobacterium avium
  misc_feature (1)..(20): Primer F7
cattcagaatcgctgcaatc

SEQ ID NO: 18 - DNA, 20 bp, Mycobacterium avium
  misc_feature (1)..(20): Primer F8
tggcgtcagctattggtgta

SEQ ID NO: 19 - DNA, 20 bp, Mycobacterium avium
  misc_feature (1)..(20): Probe F7F8
aactcgaacacacctgggac

SEQ ID NO: 20 - DNA, 20 bp, Mycobacterium avium
  misc_feature (1)..(20): Primer F9
agaatcgctgcaatctcagg

SEQ ID NO: 21 - DNA, 20 bp, Mycobacterium avium
  misc_feature (1)..(20): Primer F10
tggcgtcagctattggtgta

SEQ ID NO: 22 - DNA, 20 bp, Mycobacterium avium
  misc_feature (1)..(20): Probe F9F10
aactcgaacacacctgggac

SEQ ID NO: 23 - DNA, 20 bp, Mycobacterium avium
  misc_feature (1)..(20): Primer M1
cgaatcgcgttacatcacag

SEQ ID NO: 24 - DNA, 20 bp, Mycobacterium avium
  misc_feature (1)..(20): Primer M2
gaaaccacgttgcgagtacc

SEQ ID NO: 25 - DNA, 20 bp, Mycobacterium avium
  misc_feature (1)..(20): Probe M1M2
taccgactgagctacctggc

SEQ ID NO: 26 - DNA, 20 bp, Mycobacterium avium
  misc_feature (1)..(20): Primer M3
atcacaggtcttccggtcac

SEQ ID NO: 27 - DNA, 20 bp, Mycobacterium avium
  misc_feature (1)..(20): Primer M4
gaaaccacgttgcgagtacc

SEQ ID NO: 28 - DNA, 20 bp, Mycobacterium avium
  misc_feature (1)..(20): Probe M3M4
taccgactgagctacctggc

SEQ ID NO: 29 - DNA, 20 bp, Mycobacterium avium
  misc_feature (1)..(20): Primer M5
gacgaatcgcgttacatcac

SEQ ID NO: 30 - DNA, 20 bp, Mycobacterium avium
  misc_feature (1)..(20): Primer M6
gaaaccacgttgcgagtacc

SEQ ID NO: 31 - DNA, 20 bp, Mycobacterium avium
  misc_feature (1)..(20): Probe M5M6
taccgactgagctacctggc

SEQ ID NO: 32 - DNA, 20 bp, Mycobacterium avium
  misc_feature (1)..(20): Primer M7
tcgcgttacatcacaggtct

SEQ ID NO: 33 - DNA, 20 bp, Mycobacterium avium
  misc_feature (1)..(20): Primer M8
gaaaccacgttgcgagtacc

SEQ ID NO: 34 - DNA, 20 bp, Mycobacterium avium
  misc_feature (1)..(20): Probe M7M8
taccgactgagctacctggc

SEQ ID NO: 35 - DNA, 20 bp, Mycobacterium avium
  misc_feature (1)..(20): Primer M9
gaatcgcgttacatcacagg

SEQ ID NO: 36 - DNA, 20 bp, Mycobacterium avium
  misc_feature (1)..(20): Primer M10
gaaaccacgttgcgagtacc

SEQ ID NO: 37 - DNA, 20 bp, Mycobacterium avium
  misc_feature (1)..(20): Probe M9M10
taccgactgagctacctggc

SEQ ID NO: 38 - DNA, 20 bp, Mycobacterium avium
  misc_feature (1)..(20): Primer F1F2N1
gtcattcagaatcgctgcaa

SEQ ID NO: 39 - DNA, 21 bp, Mycobacterium avium
  misc_feature (1)..(21): Primer F1F2N2
cgtggtctctgagtttgggta

SEQ ID NO: 40 - DNA, 20 bp, Mycobacterium avium
  misc_feature (1)..(20): Probe F1F2N1F1F2N2
ctggtagacgcccatttcat

SEQ ID NO: 41 - DNA, 20 bp, Mycobacterium avium
  misc_feature (1)..(20): Primer F1F2N3
gtcattcagaatcgctgcaa

SEQ ID NO: 42 - DNA, 20 bp, Mycobacterium avium
  misc_feature (1)..(20): Primer F1F2N4
tatcgatgaaatgggcgtct

SEQ ID NO: 43 - DNA, 20 bp, Mycobacterium avium
  misc_feature (1)..(20): Probe F1F2N3F1F2N4
cagctccagatcgtcattca

SEQ ID NO: 44 - DNA, 20 bp, Mycobacterium avium
  misc_feature (1)..(20): Primer F1F2N5
gtcattcagaatcgctgcaa

SEQ ID NO: 45 - DNA, 21 bp, Mycobacterium avium
  misc_feature (1)..(21): Primer F1F2N6
ccactcgtggtctctgagttt

SEQ ID NO: 46 - DNA, 20 bp, Mycobacterium avium
  misc_feature (1)..(20): Probe F1F2N5F1F2N6
ctggtagacgcccatttcat

SEQ ID NO: 47 - DNA, 20 bp, Mycobacterium avium
  misc_feature (1)..(20): Primer F1F2N7
gtcattcagaatcgctgcaa

SEQ ID NO: 48 - DNA, 20 bp, Mycobacterium avium
  misc_feature (1)..(20): Primer F1F2N8
atcgatgaaatgggcgtcta

SEQ ID NO: 49 - DNA, 20 bp, Mycobacterium avium
  misc_feature (1)..(20): Probe F1F2N7F1F2N8
cagctccagatcgtcattca

SEQ ID NO: 50 - DNA, 20 bp, Mycobacterium avium
  misc_feature (1)..(20): Primer F1F2N9
gtcattcagaatcgctgcaa

SEQ ID NO: 51 - DNA, 20 bp, Mycobacterium avium
  misc_feature (1)..(20): Primer F1F2N10
ctcgtggtctctgagtttgg

SEQ ID NO: 52 - DNA, 20 bp, Mycobacterium avium
  misc_feature (1)..(20): Probe F1F2N9F1F2N10
ctggtagacgcccatttcat

SEQ ID NO: 53 - DNA, 20 bp, Mycobacterium avium
  misc_feature (1)..(20): Primer F3F4N1
gtcattcagaatcgctgcaa

SEQ ID NO: 54 - DNA, 21 bp, Mycobacterium avium
  misc_feature (1)..(21): Primer F3F4N2
cgtggtctctgagtttgggta

SEQ ID NO: 55 - DNA, 20 bp, Mycobacterium avium
  misc_feature (1)..(20): Probe F3F4N1F3F4N2
ctggtagacgcccatttcat

SEQ ID NO: 56 - DNA, 20 bp, Mycobacterium avium
  misc_feature (1)..(20): Primer F3F4N3
gtcattcagaatcgctgcaa

SEQ ID NO: 57 - DNA, 20 bp, Mycobacterium avium
  misc_feature (1)..(20): Primer F3F4N4
tatcgatgaaatgggcgtct

SEQ ID NO: 58 - DNA, 20 bp, Mycobacterium avium
  misc_feature (1)..(20): Probe F3F4N3F3F4N4
cagctccagatcgtcattca

SEQ ID NO: 59 - DNA, 20 bp, Mycobacterium avium
  misc_feature (1)..(20): Primer F3F4N5
gtcattcagaatcgctgcaa

SEQ ID NO: 60 - DNA, 21 bp, Mycobacterium avium
  misc_feature (1)..(21): Primer F3F4N6
ccactcgtggtctctgagttt

SEQ ID NO: 61 - DNA, 20 bp, Mycobacterium avium
  misc_feature (1)..(20): Probe F3F4N5F3F4N6
ctggtagacgcccatttcat

SEQ ID NO: 62 - DNA, 20 bp, Mycobacterium avium
  misc_feature (1)..(20): Primer F3F4N7
gtcattcagaatcgctgcaa

SEQ ID NO: 63 - DNA, 20 bp, Mycobacterium avium
  misc_feature (1)..(20): Primer F3F4N8
atcgatgaaatgggcgtcta

SEQ ID NO: 64 - DNA, 20 bp, Mycobacterium avium
  misc_feature (1)..(20): Probe F3F4N7F3F4N8
cagctccagatcgtcattca

SEQ ID NO: 65 - DNA, 20 bp, Mycobacterium avium
  misc_feature (1)..(20): Primer F3F4N9
gtcattcagaatcgctgcaa

SEQ ID NO: 66 - DNA, 20 bp, Mycobacterium avium
  misc_feature (1)..(20): Primer F3F4N10
ctcgtggtctctgagtttgg

SEQ ID NO: 67 - DNA, 20 bp, Mycobacterium avium
  misc_feature (1)..(20): Probe F3F4N9F3F4N10
ctggtagacgcccatttcat

SEQ ID NO: 68 - DNA, 20 bp, Mycobacterium avium
  misc_feature (1)..(20): Primer F5F6N1
agaatcgctgcaatctcagg

SEQ ID NO: 69 - DNA, 21 bp, Mycobacterium avium
  misc_feature (1)..(21): Primer F5F6N2
cgtggtctctgagtttgggta

SEQ ID NO: 70 - DNA, 19 bp, Mycobacterium avium
  misc_feature (1)..(19): Probe F5F6N1F5F6N2
cgcttgaatggtcgtctgt

SEQ ID NO: 71 - DNA, 20 bp, Mycobacterium avium
  misc_feature (1)..(20): Primer F5F6N3
agaatcgctgcaatctcagg

SEQ ID NO: 72 - DNA, 20 bp, Mycobacterium avium
  misc_feature (1)..(20): Primer F5F6N4
cttagttcgccgcttgaatg

SEQ ID NO: 73 - DNA, 20 bp, Mycobacterium avium
  misc_feature (1)..(20): Probe F5F6N3F5F6N4
ctggtagacgcccatttcat

SEQ ID NO: 74 - DNA, 20 bp, Mycobacterium avium
  misc_feature (1)..(20): Primer F5F6N5
agaatcgctgcaatctcagg

SEQ ID NO: 75 - DNA, 21 bp, Mycobacterium avium
  misc_feature (1)..(21): Primer F5F6N6
ccactcgtggtctctgagttt

SEQ ID NO: 76 - DNA, 20 bp, Mycobacterium avium
  misc_feature (1)..(20): Probe F5F6N5F5F6N6
ctggtagacgcccatttcat

SEQ ID NO: 77 - DNA, 19 bp, Mycobacterium avium
  misc_feature (1)..(19): Primer F5F6N7
ctgcaatctcaggcagctc

SEQ ID NO: 78 - DNA, 20 bp, Mycobacterium avium
  misc_feature (1)..(20): Primer F5F6N8
cttagttcgccgcttgaatg

SEQ ID NO: 79 - DNA, 20 bp, Mycobacterium avium
  misc_feature (1)..(20): Probe F5F6N7F5F6N8
ctggtagacgcccatttcat

SEQ ID NO: 80 - DNA, 19 bp, Mycobacterium avium
  misc_feature (1)..(19): Primer F5F6N9
ctgcaatctcaggcagctc

SEQ ID NO: 81 - DNA, 19 bp, Mycobacterium avium
  misc_feature (1)..(19): Primer F5F6N10
ttagttcgccgcttgaatg

SEQ ID NO: 82 - DNA, 20 bp, Mycobacterium avium
  misc_feature (1)..(20): Probe F5F6N9F5F6N10
ctggtagacgcccatttcat

SEQ ID NO: 83 - DNA, 20 bp, Mycobacterium avium
  misc_feature (1)..(20): Primer F7F8N1
cagctccagatcgtcattca

SEQ ID NO: 84 - DNA, 19 bp, Mycobacterium avium
  misc_feature (1)..(19): Primer F7F8N2
tgtcgatccgcttagttcg

SEQ ID NO: 85 - DNA, 20 bp, Mycobacterium avium
  misc_feature (1)..(20): Probe F7F8N1F7F8N2
ctggtagacgcccatttcat

SEQ ID NO: 86 - DNA, 20 bp, Mycobacterium avium
  misc_feature (1)..(20): Primer F7F8N3
gcattccaagtcctgaccac

SEQ ID NO: 87 - DNA, 20 bp, Mycobacterium avium
  misc_feature (1)..(20): Primer F7F8N4
gtcccaggtgtgttcgagtt

SEQ ID NO: 88 - DNA, 20 bp, Mycobacterium avium
  misc_feature (1)..(20): Probe F7F8N3F7F8N4
ctggtagacgcccatttcat

SEQ ID NO: 89 - DNA, 20 bp, Mycobacterium avium
  misc_feature (1)..(20): Primer F7F8N5
cagctccagatcgtcattca

SEQ ID NO: 90 - DNA, 20 bp, Mycobacterium avium
  misc_feature (1)..(20): Primer F7F8N6
ttgtcgatccgcttagttcg

SEQ ID NO: 91 - DNA, 20 bp, Mycobacterium avium
  misc_feature (1)..(20): Probe F7F8N5F7F8N6
ctggtagacgcccatttcat

SEQ ID NO: 92 - DNA, 20 bp, Mycobacterium avium
  misc_feature (1)..(20): Primer F7F8N7
agaatcgctgcaatctcagg

SEQ ID NO: 93 - DNA, 19 bp, Mycobacterium avium
  misc_feature (1)..(19): Primer F7F8N8
cgcttgaatggtcgtctgt

SEQ ID NO: 94 - DNA, 20 bp, Mycobacterium avium
  misc_feature (1)..(20): Probe F7F8N7F7F8N8
ctggtagacgcccatttcat

SEQ ID NO: 95 - DNA, 20 bp, Mycobacterium avium
  misc_feature (1)..(20): Primer F7F8N9
agaatcgctgcaatctcagg

SEQ ID NO: 96 - DNA, 20 bp, Mycobacterium avium
  misc_feature (1)..(20): Primer F7F8N10
cttagttcgccgcttgaatg

SEQ ID NO: 97 - DNA, 20 bp, Mycobacterium avium
  misc_feature (1)..(20): Probe F7F8N9F7F8N10
ctggtagacgcccatttcat

SEQ ID NO: 98 - DNA, 20 bp, Mycobacterium avium
  misc_feature (1)..(20): Primer F9F10N1
cagctccagatcgtcattca

SEQ ID NO: 99 - DNA, 19 bp, Mycobacterium avium
  misc_feature (1)..(19): Primer F9F10N2
tgtcgatccgcttagttcg

SEQ ID NO: 100 - DNA, 20 bp, Mycobacterium avium
  misc_feature (1)..(20): Probe F9F10N1F9F10N2
ctggtagacgcccatttcat

SEQ ID NO: 101 - DNA, 20 bp, Mycobacterium avium
  misc_feature (1)..(20): Primer F9F10N3
cagctccagatcgtcattca

SEQ ID NO: 102 - DNA, 20 bp, Mycobacterium avium
  misc_feature (1)..(20): Primer F9F10N4
ttgtcgatccgcttagttcg

SEQ ID NO: 103 - DNA, 20 bp, Mycobacterium avium
  misc_feature (1)..(20): Probe F9F10N3F9F10N4
ctggtagacgcccatttcat

SEQ ID NO: 104 - DNA, 20 bp, Mycobacterium avium
  misc_feature (1)..(20): Primer F9F10N5
gcattccaagtcctgaccac

SEQ ID NO: 105 - DNA, 20 bp, Mycobacterium avium
  misc_feature (1)..(20): Primer F9F10N6
caggtgtgttcgagttgcag

SEQ ID NO: 106 - DNA, 20 bp, Mycobacterium avium
  misc_feature (1)..(20): Probe F9F10N5F9F10N6
ctggtagacgcccatttcat

SEQ ID NO: 107 - DNA, 20 bp, Mycobacterium avium
  misc_feature (1)..(20): Primer F9F10N7
gcagctccagatcgtcattc

SEQ ID NO: 108 - DNA, 19 bp, Mycobacterium avium
  misc_feature (1)..(19): Primer F9F10N8
tgtcgatccgcttagttcg

SEQ ID NO: 109 - DNA, 20 bp, Mycobacterium avium
  misc_feature (1)..(20): Probe F9F10N7F9F10N8
ctggtagacgcccatttcat

SEQ ID NO: 110 - DNA, 20 bp, Mycobacterium avium
  misc_feature (1)..(20): Primer F9F10N9
cagctccagatcgtcattca

SEQ ID NO: 111 - DNA, 21 bp, Mycobacterium avium
  misc_feature (1)..(21): Primer F9F10N10
tgagaattgtcgatccgctta

SEQ ID NO: 112 - DNA, 20 bp, Mycobacterium avium
  misc_feature (1)..(20): Probe F9F10N9F9F10N10
ctggtagacgcccatttcat

SEQ ID NO: 113 - DNA, 19 bp, Mycobacterium avium
  misc_feature (1)..(19): Primer M1M2N1
ggcagcatgctcaagtagc

SEQ ID NO: 114 - DNA, 18 bp, Mycobacterium avium
  misc_feature (1)..(18): Primer M1M2N2
gggttcgaatcccgtagg

SEQ ID NO: 115 - DNA, 20 bp, Mycobacterium avium
  misc_feature (1)..(20): Probe M1M2N1M1M2N2
taccgactgagctacctggc

SEQ ID NO: 116 - DNA, 19 bp, Mycobacterium avium
  misc_feature (1)..(19): Primer M1M2N3
gcagcatgctcaagtagcc

SEQ ID NO: 117 - DNA, 18 bp, Mycobacterium avium
  misc_feature (1)..(18): Primer M1M2N4
gggttcgaatcccgtagg

SEQ ID NO: 118 - DNA, 20 bp, Mycobacterium avium
  misc_feature (1)..(20): Probe M1M2N3M1M2N4
taccgactgagctacctggc

SEQ ID NO: 119 - DNA, 19 bp, Mycobacterium avium
  misc_feature (1)..(19): Primer M1M2N5
gcagcatgctcaagtagcc

SEQ ID NO: 120 - DNA, 18 bp, Mycobacterium avium
  misc_feature (1)..(18): Primer M1M2N6
ccctttcaaggcggtagc

SEQ ID NO: 121 - DNA, 20 bp, Mycobacterium avium
  misc_feature (1)..(20): Probe M1M2N5M1M2N6
taccgactgagctacctggc

SEQ ID NO: 122 - DNA, 19 bp, Mycobacterium avium
  misc_feature (1)..(19): Primer M1M2N7
gcagcatgctcaagtagcc

SEQ ID NO: 123 - DNA, 18 bp, Mycobacterium avium
  misc_feature (1)..(18): Primer M1M2N8
gccctttcaaggcggtag

SEQ ID NO: 124 - DNA, 20 bp, Mycobacterium avium
  misc_feature (1)..(20): Probe M1M2N7M1M2N8
taccgactgagctacctggc

SEQ ID NO: 125 - DNA, 19 bp, Mycobacterium avium
  misc_feature (1)..(19): Primer M1M2N9
ggcagcatgctcaagtagc

SEQ ID NO: 126 - DNA, 18 bp, Mycobacterium avium
  misc_feature (1)..(18): Primer M1M2N10
ccctttcaaggcggtagc

SEQ ID NO: 127 - DNA, 20 bp, Mycobacterium avium
  misc_feature (1)..(20): Probe M1M2N9M1M2N10
taccgactgagctacctggc

SEQ ID NO: 128 - DNA, 19 bp, Mycobacterium avium
  misc_feature (1)..(19): Primer M3M4N1
ggcagcatgctcaagtagc

SEQ ID NO: 129 - DNA, 18 bp, Mycobacterium avium
  misc_feature (1)..(18): Primer M3M4N2
gggttcgaatcccgtagg

SEQ ID NO: 130 - DNA, 20 bp, Mycobacterium avium
  misc_feature (1)..(20): Probe M3M4N1M3M4N2
taccgactgagctacctggc

SEQ ID NO: 131 - DNA, 19 bp, Mycobacterium avium
  misc_feature (1)..(19): Primer M3M4N3
gcagcatgctcaagtagcc

SEQ ID NO: 132 - DNA, 18 bp, Mycobacterium avium
  misc_feature (1)..(18): Primer M3M4N4
gggttcgaatcccgtagg

SEQ ID NO: 133 - DNA, 20 bp, Mycobacterium avium
  misc_feature (1)..(20): Probe M3M4N3M3M4N4
taccgactgagctacctggc

SEQ ID NO: 134 - DNA, 19 bp, Mycobacterium avium
  misc_feature (1)..(19): Primer M3M4N5
gcagcatgctcaagtagcc

SEQ ID NO: 135 - DNA, 18 bp, Mycobacterium avium
  misc_feature (1)..(18): Primer M3M4N6
ccctttcaaggcggtagc

SEQ ID NO: 136 - DNA, 20 bp, Mycobacterium avium
  misc_feature (1)..(20): Probe M3M4N5M3M4N6
taccgactgagctacctggc

SEQ ID NO: 137 - DNA, 19 bp, Mycobacterium avium
  misc_feature (1)..(19): Primer M3M4N7
gcagcatgctcaagtagcc

SEQ ID NO: 138 - DNA, 18 bp, Mycobacterium avium
  misc_feature (1)..(18): Primer M3M4N8
gccctttcaaggcggtag

SEQ ID NO: 139 - DNA, 20 bp, Mycobacterium avium
  misc_feature (1)..(20): Probe M3M4N7M3M4N8
taccgactgagctacctggc

SEQ ID NO: 140 - DNA, 19 bp, Mycobacterium avium
  misc_feature (1)..(19): Primer M3M4N9
ggcagcatgctcaagtagc

SEQ ID NO: 141 - DNA, 18 bp, Mycobacterium avium
  misc_feature (1)..(18): Primer M3M4N10
ccctttcaaggcggtagc

SEQ ID NO: 142 - DNA, 20 bp, Mycobacterium avium
  misc_feature (1)..(20): Probe M3M4N9M3M4N10
taccgactgagctacctggc

SEQ ID NO: 143 - DNA, 19 bp, Mycobacterium avium
  misc_feature (1)..(19): Primer M5M6N1
ggcagcatgctcaagtagc

SEQ ID NO: 144 - DNA, 19 bp, Mycobacterium avium
  misc_feature (1)..(19): Primer M5M6N2
ctgtggcgcagttggttag

SEQ ID NO: 145 - DNA, 20 bp, Mycobacterium avium
  misc_feature (1)..(20): Probe M5M6N1M5M6N2
taccgactgagctacctggc

SEQ ID NO: 146 - DNA, 19 bp, Mycobacterium avium
  misc_feature (1)..(19): Primer M5M6N3
gcagcatgctcaagtagcc

SEQ ID NO: 147 - DNA, 19 bp, Mycobacterium avium
  misc_feature (1)..(19): Primer M5M6N4
ctgtggcgcagttggttag

SEQ ID NO: 148 - DNA, 20 bp, Mycobacterium avium
  misc_feature (1)..(20): Probe M5M6N3M5M6N4
taccgactgagctacctggc

SEQ ID NO: 149 - DNA, 19 bp, Mycobacterium avium
  misc_feature (1)..(19): Primer M5M6N5
cggcagcatgctcaagtag

SEQ ID NO: 150 - DNA, 19 bp, Mycobacterium avium
  misc_feature (1)..(19): Primer M5M6N6
ctgtggcgcagttggttag

SEQ ID NO: 151 - DNA, 20 bp, Mycobacterium avium
  misc_feature (1)..(20): Probe M5M6N5M5M6N6
taccgactgagctacctggc

SEQ ID NO: 152 - DNA, 18 bp, Mycobacterium avium
  misc_feature (1)..(18): Primer M5M6N7
cggcagcatgctcaagta

SEQ ID NO: 153 - DNA, 19 bp, Mycobacterium avium
  misc_feature (1)..(19): Primer M5M6N8
ctgtggcgcagttggttag

SEQ ID NO: 154 - DNA, 20 bp, Mycobacterium avium
  misc_feature (1)..(20): Probe M5M6N7M5M6N8
taccgactgagctacctggc

SEQ ID NO: 155 - DNA, 19 bp, Mycobacterium avium
  misc_feature (1)..(19): Primer M5M6N9
ggcagcatgctcaagtagc

SEQ ID NO: 156 - DNA, 18 bp, Mycobacterium avium
  misc_feature (1)..(18): Primer M5M6N10
gtggcgcagttggttagc

SEQ ID NO: 157 - DNA, 20 bp, Mycobacterium avium
  misc_feature (1)..(20): Probe M5M6N9M5M6N10
taccgactgagctacctggc

SEQ ID NO: 158 - DNA, 19 bp, Mycobacterium avium
  misc_feature (1)..(19): Primer M7M8N1
ggcagcatgctcaagtagc

SEQ ID NO: 159 - DNA, 18 bp, Mycobacterium avium
  misc_feature (1)..(18): Primer M7M8N2
gggttcgaatcccgtagg

SEQ ID NO: 160 - DNA, 20 bp, Mycobacterium avium
  misc_feature (1)..(20): Probe M7M8N1M7M8N2
taccgactgagctacctggc

SEQ ID NO: 161 - DNA, 19 bp, Mycobacterium avium
  misc_feature (1)..(19): Primer M7M8N3
gcagcatgctcaagtagcc

SEQ ID NO: 162 - DNA, 18 bp, Mycobacterium avium
  misc_feature (1)..(18): Primer M7M8N4
gggttcgaatcccgtagg

SEQ ID NO: 163 - DNA, 20 bp, Mycobacterium avium
  misc_feature (1)..(20): Probe M7M8N3M7M8N4
taccgactgagctacctggc

SEQ ID NO: 164 - DNA, 19 bp, Mycobacterium avium
  misc_feature (1)..(19): Primer M7M8N5
gcagcatgctcaagtagcc

SEQ ID NO: 165 - DNA, 18 bp, Mycobacterium avium
  misc_feature (1)..(18): Primer M7M8N6
ccctttcaaggcggtagc

SEQ ID NO: 166 - DNA, 20 bp, Mycobacterium avium
  misc_feature (1)..(20): Probe M7M8N5M7M8N6
taccgactgagctacctggc

SEQ ID NO: 167 - DNA, 19 bp, Mycobacterium avium
  misc_feature (1)..(19): Primer M7M8N7
gcagcatgctcaagtagcc

SEQ ID NO: 168 - DNA, 18 bp, Mycobacterium avium
  misc_feature (1)..(18): Primer M7M8N8
gccctttcaaggcggtag

SEQ ID NO: 169 - DNA, 20 bp, Mycobacterium avium
  misc_feature (1)..(20): Probe M7M8N7M7M8N8
taccgactgagctacctggc

SEQ ID NO: 170 - DNA, 19 bp, Mycobacterium avium
  misc_feature (1)..(19): Primer M7M8N9
ggcagcatgctcaagtagc

SEQ ID NO: 171 - DNA, 18 bp, Mycobacterium avium
  misc_feature (1)..(18): Primer M7M8N10
ccctttcaaggcggtagc

SEQ ID NO: 172 - DNA, 20 bp, Mycobacterium avium
  misc_feature (1)..(20): Probe M7M8N9M7M8N10
taccgactgagctacctggc

SEQ ID NO: 173 - DNA, 19 bp, Mycobacterium avium
  misc_feature (1)..(19): Primer M9M10N1
gcagcatgctcaagtagcc

SEQ ID NO: 174 - DNA, 18 bp, Mycobacterium avium
  misc_feature (1)..(18): Primer M9M10N2
aatcccgtagggggtacg

SEQ ID NO: 175 - DNA, 20 bp, Mycobacterium avium
  misc_feature (1)..(20): Probe M9M10N1M9M10N2
taccgactgagctacctggc

SEQ ID NO: 176 - DNA, 19 bp, Mycobacterium avium
  misc_feature (1)..(19): Primer M9M10N3
ggcagcatgctcaagtagc

SEQ ID NO: 177 - DNA, 18 bp, Mycobacterium avium
  misc_feature (1)..(18): Primer M9M10N4
aatcccgtagggggtacg

SEQ ID NO: 178 - DNA, 20 bp, Mycobacterium avium
  misc_feature (1)..(20): Probe M9M10N3M9M10N4
taccgactgagctacctggc

SEQ ID NO: 179 - DNA, 19 bp, Mycobacterium avium
  misc_feature (1)..(19): Primer M9M10N5
gcagcatgctcaagtagcc

SEQ ID NO: 180 - DNA, 19 bp, Mycobacterium avium
  misc_feature (1)..(19): Primer M9M10N6
gaatcccgtagggggtacg

SEQ ID NO: 181 - DNA, 20 bp, Mycobacterium avium
  misc_feature (1)..(20): Probe M9M10N5M9M10N6
taccgactgagctacctggc

SEQ ID NO: 182 - DNA, 19 bp, Mycobacterium avium
  misc_feature (1)..(19): Primer M9M10N7
ggcagcatgctcaagtagc

SEQ ID NO: 183 - DNA, 19 bp, Mycobacterium avium
  misc_feature (1)..(19): Primer M9M10N8
gaatcccgtagggggtacg

SEQ ID NO: 184 - DNA, 20 bp, Mycobacterium avium
  misc_feature (1)..(20): Probe M9M10N7M9M10N8
taccgactgagctacctggc

SEQ ID NO: 185 - DNA, 19 bp, Mycobacterium avium
  misc_feature (1)..(19): Primer M9M10N9
gcagcatgctcaagtagcc

SEQ ID NO: 186 - DNA, 18 bp, Mycobacterium avium
  misc_feature (1)..(18): Primer M9M10N10
gggttcgaatcccgtagg

SEQ ID NO: 187 - DNA, 20 bp, Mycobacterium avium
  misc_feature (1)..(20): Probe M9M10N9M9M10N10
taccgactgagctacctggc

SEQ ID NO: 188 - DNA, 20 bp, Mycobacterium avium
  misc_feature (1)..(20): Primer P901
ggcacggctcttgttgtagt

SEQ ID NO: 189 - DNA, 18 bp, Mycobacterium avium
  misc_feature (1)..(18): Primer P902
gcgctgctggagttgatt

SEQ ID NO: 190 - DNA, 20 bp, Mycobacterium avium
  misc_feature (1)..(20): Probe P901P902
gaatataaagcagccgctgc

SEQ ID NO: 191 - DNA, 20 bp, Mycobacterium avium
  misc_feature (1)..(20): Primer P901A
cacggctcttgttgtagtcg

SEQ ID NO: 192 - DNA, 18 bp, Mycobacterium avium
  misc_feature (1)..(18): Primer P902A
gcgctgctggagttgatt

SEQ ID NO: 193 - DNA, 20 bp, Mycobacterium avium
  misc_feature (1)..(20): Probe P901AP902A
gaatataaagcagccgctgc

SEQ ID NO: 194 - DNA, 20 bp, Mycobacterium avium
  misc_feature (1)..(20): Primer P901B
cggctcttgttgtagtcgaa

SEQ ID NO: 195 - DNA, 18 bp, Mycobacterium avium
  misc_feature (1)..(18): Primer P902B
gcgctgctggagttgatt

SEQ ID NO: 196 - DNA, 20 bp, Mycobacterium avium
  misc_feature (1)..(20): Probe P901BP902B
gaatataaagcagccgctgc

SEQ ID NO: 197 - DNA, 21 bp, Mycobacterium avium
  misc_feature (1)..(21): Primer P901C
cggctcttgttgtagtcgaag

SEQ ID NO: 198 - DNA, 18 bp, Mycobacterium avium
  misc_feature (1)..(18): Primer P902C
gcgctgctggagttgatt

SEQ ID NO: 199 - DNA, 20 bp, Mycobacterium avium
  misc_feature (1)..(20): Probe P901CP902C
gaatataaagcagccgctgc

SEQ ID NO: 200 - DNA, 21 bp, Mycobacterium avium
  misc_feature (1)..(21): Primer P901D
acggctcttgttgtagtcgaa

SEQ ID NO: 201 - DNA, 18 bp, Mycobacterium avium
  misc_feature (1)..(18): Primer P902D
gcgctgctggagttgatt

SEQ ID NO: 202 - DNA, 20 bp, Mycobacterium avium
  misc_feature (1)..(20): Probe P901DP902D
gaatataaagcagccgctgc

SEQ ID NO: 203 - DNA, 19 bp, Mycobacterium avium
  misc_feature (1)..(19): Primer P901N
gttccagcgccgaaagtat

SEQ ID NO: 204 - DNA, 19 bp, Mycobacterium avium
  misc_feature (1)..(19): Primer P902N
caagaccgacgccaaagac

SEQ ID NO: 205 - DNA, 19 bp, Mycobacterium avium
  misc_feature (1)..(19): Primer P901AN
gttccagcgccgaaagtat

SEQ ID NO: 206 - DNA, 18 bp, Mycobacterium avium
  misc_feature (1)..(18): Primer P902AN
caagaccgacgccaaaga

SEQ ID NO: 207 - DNA, 20 bp, Mycobacterium avium
  misc_feature (1)..(20): Primer P901BN
gttccagcgccgaaagtatt

SEQ ID NO: 208 - DNA, 19 bp, Mycobacterium avium
  misc_feature (1)..(19): Primer P902BN
caagaccgacgccaaagac

SEQ ID NO: 209 - DNA, 19 bp, Mycobacterium avium
  misc_feature (1)..(19): Primer P901CN
agcgccgaaagtattccag

SEQ ID NO: 210 - DNA, 19 bp, Mycobacterium avium
  misc_feature (1)..(19): Primer P902CN
caagaccgacgccaaagac

SEQ ID NO: 211 - DNA, 20 bp, Mycobacterium avium
  misc_feature (1)..(20): Primer P901DN
gttccagcgccgaaagtatt

SEQ ID NO: 212 - DNA, 18 bp, Mycobacterium avium
  misc_feature (1)..(18): Primer P902DN
caagaccgacgccaaaga

SEQ ID NO: 213 - DNA, 620 bp, Mycobacterium avium
  misc_feature (1)..(620): Mycobacterium avium subspecies M. Paratuberculosis species specific F57 DNA fragment
ggatctcggccccgatagctttcctctccttcgtcaccaactggcgcgggtccaggaacgcttggcactcgtcaatcaccacgaacaccagcggaacctgcggcgtcgggccgcgatcccaaaagttggacgatccgaatatgttgttgccctgtctaattcgatcacggactagaccggtcgcgtcattcagaatcgctgcaatctcaggcagctccagatcgtcattcatgaatcggcagctacgagcacgcaggcattccaagtcctgaccacccttcccgtcgatgacagcgaactggaccgccgctgacgcaccgaacgaccccagagcactcgtcagccacgcggttttccccgaccctggtacgcctcccactacaacgcccgatacgttcgccaacgggagcgactggtagacgcccatttcatcgatacccaaactcagagaccacgagtgggcggctggcacagacgaccattcaagcggcgaactaagcggatcgacaattctcagctgcaactcgaacacacctgggaccggctcggtgacaacaacattcggtacaccaatagctgacgccagcttcggagccgccacctgccactgtctgagatcc

(19) United States
(12) Patent Application Publication
(10) Pub. No.: US 2010/0021897 A1
(43) Pub. Date: Jan. 28, 2010

(54) Mycobacterium Avium Subspecies Paratuberculosis (Map) Diagnostic Test

(76) Inventors: Joseph Elliot Williams, Newberry, FL (US); Gilles R.G. Monif, Bellevue, NE (US); Claus D. Buergelt, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/440,795

(22) PCT Filed: Sep. 11, 2007
(86) PCT No.: PCT/US07/78117

§ 371 Date: Sep. 22, 2009

Related U.S. Application Data
(60) Provisional application No. 60/843,865, filed on Sep. 11, 2006.
(60) Provisional application No. 60/951,357, filed on Jul. 23, 2007.

(51) Int. Cl. C12Q 1/68; C07H 21/04

(52) U.S. Cl. 435/6; 536/24.33; 536/23.1

(57) ABSTRACT

The subject invention provides PCR primers and other nucleic acid sequences derived from Mycobacterium avium genes (IS1311 (Genbank accession # U16276), F57 (Accession # X70277) and ISMav2 (Accession # AF286339); these accession numbers and their respective descriptions are hereby incorporated by reference in their entirety and are provided in SEQ ID NOs: 5, 7 and 213). Additionally, the subject invention also provides methods of identifying animals or individuals infected with Mycobacterium avium subsp. paratuberculosis (MAP). Non-limiting examples of identifying such animals or individuals include various nucleic hybridization techniques such as, and not limited to, Northern blots, Southern blots and PCR techniques.

### CROSS-REFERENCE TO RELATED APPLICATIONS

This application claims the benefit of U.S. Provisional Application Ser. No. 60/843,865, filed Sep. 11, 2006 and U.S. Provisional Application Ser. No. 60/951,357, filed Jul. 23, 2007, the disclosures of which are hereby incorporated by reference in their entirety, including all figures, tables and amino acid or nucleic acid sequences.

### BACKGROUND OF THE INVENTION

Paratuberculosis (Johne's disease) caused by Mycobacterium avium subsp. paratuberculosis (MAP), a facultative intracellular, acid-fast bacillus, affects ruminants worldwide. In the United States the disease causes the industry economic losses estimated at $200 and 250 million. The control of the disease is hampered by ineffective diagnostic methods, particularly in detection of infected, sub-clinical animals.

A fragment of infected animals in a given herd can be presumptively diagnosed based on clinical signs of diarrhea and emaciation and serology. The animals can be reliably diagnosed with conventional and/or radiometric fecal culture. With infected, sub-clinical animals, agent detection or serology frequently lead to false negative results. As there are no methods currently available for the management of paratuberculosis such as treatment or vaccination, farmers depend on test and cull programs to control the disease.

Several methods for screening for the presence of MAP in tissue samples from affected animals are known. Commonly used immunological methods for detecting MAP in a sample include agar gel immunodiffusion (AGID) tests and ELISA assays. More rapid DNA-based tests have been developed that utilize PCR in conjunction with pairs of primers that specifically detect species-specific insertion sequences present in MAP strains, but not in other strains of Mycobacterium avium. A commercial DNA-based assay is available for detecting a 413 bp PCR product amplified from the MAP insertion sequence defined as IS900 (Vary, P. H. et al., J Clin Microbiol 1990; 28:933-937). While useful, previous PCR-based methods of diagnosing MAP infection were not reliably sensitive enough to detect subclinical MAP infections. Thus, to better prevent the spread of MAP infection in a herd of animals, a more sensitive PCR-based MAP detection method would be useful. Methods that allow reliable detection of preclinical infection would be especially desirable.

The current “gold standard” method for diagnosis of sub-clinical MAP infection is been based upon fecal recovery of live MAP using artificial culture media. Beckton-Dickinson Biosciences has recently developed an automated system (BACTEC MGIT 960 system) that can be used as a fully automated diagnostic tool for Johne's disease. Although this technique is highly specific, it is still suboptimal in terms of sensitivity. Additionally, culture from a fecal sample is only deemed negative after 49 days. This however is an imperfect diagnosis because cultures may become positive as long as six months after inoculation. In very rare instances, cultures have been reputed to become positive between six months and one year. Due to the amount of time a sample must be cultured, the expense of the specialized culture reagents and the BACTEC MGIT 960 system, this test is expensive. The cost to process a single sample ranges from $16.00 to $45.00 (depending upon the degree to which a given state subsidizes testing costs). The subject application provides materials and methods for the reliable and relatively inexpensive identification of MAP infections, including subclinical infections, in animals or other individuals.

### BRIEF SUMMARY OF THE INVENTION

The subject invention provides PCR primers and other nucleic acid sequences derived from Mycobacterium avium genes (IS1311 (Genbank accession # U16276), F57 (Accession # X70277) and ISMav2 (Accession # AF286339); these accession numbers and their respective descriptions are hereby incorporated by reference in their entirety and are provided in SEQ ID NOs: 5, 7 and 213). Additionally, the subject invention also provides methods of identifying animals or individuals infected with Mycobacterium avium subsp. paratuberculosis (MAP). Non-limiting examples of identifying such animals or individuals include various nucleic hybridization techniques such as, and not limited to, Northern blots, Southern blots and PCR techniques.

### BRIEF DESCRIPTION OF THE SEQUENCES SEQ ID NOs: 1-4 are primers suitable for use in PCR techniques for the identification of MAP in biological samples.

SEQ ID NO: 5 is the complete cds sequence of Mycobacterium avium subsp. paratuberculosis (MAP) and Mycobacterium avium subsp. avium (MAA) insertion sequence IS1311 transposase gene.

SEQ ID NO: 6 is the amino acid sequence of the MAP and MAA insertion sequence IS1311 transposase.

SEQ ID NO: 7 is the nucleotide sequence the Mycobacterium avium insertion sequence ISMav2 derived from the MAP genome project.

SEQ ID NOs: 8-212 are primer and probe sequences suitable for use in PCR techniques for the identification of MAP in biological samples.

SEQ ID NO: 213 is the nucleotide sequence of the Mycobacterium avium sequence F57.

### DETAILED DISCLOSURE OF THE INVENTION

The subject invention provides, in one of its various embodiments, a PCR-based method for detecting a subclinical or clinical Map infection in an animal subject by testing a biological sample. More particularly, the invention utilizes two sets of primers in a “nested PCR” method of detecting Map. Primer sets suitable for the identification of Map in biological samples are provided by the subject invention as well. One non-limiting example of such a primer set is found in two pairs of PCR primers, the first pair (IS1 (SEQ ID NO: 1) and IS2) SEQ ID NO: 2)) designed to amplify the 242 bp IS1311 sequence, and the second pair (IS3 (SEQ ID NO: 3) and IS4 (SEQ ID NO: 4)) designed to span a 104 bp region within the IS1311 region are also provided by the subject invention. These pair of primers can be used in standard or nested PCR protocols.

In the context of this invention, the term “successive” can be used interchangeably with the terms “contiguous” or “consecutive” or the phrase “contiguous span” throughout the subject application. Thus, in some embodiments, a polynucleotide fragment, probe fragment and/or primer fragment may be referred to as “a contiguous span of at least X nucleotides, wherein X is any integer value beginning with 8; the upper limit for these various fragments is one nucleotide less than the total number of nucleotides associated with a particular SEQ ID NO: provided in the Sequence Listing appended hereto (e.g., the number of nucleotides present in the polynucleotide comprising SEQ ID NO: 5 is 1317, thus a fragment of SEQ ID NO: 5 corresponds to any consecutive span of X nucleotides of SEQ ID NO: 5, wherein X is any integer between, and including, 8 and 1316).

“Nucleotide sequence”, “nucleic acids”, “polynucleotide”, “oligonucleotide” or “nucleic acid sequence” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state. The nucleic acid, polynucleotide, or nucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, subcloning or chemical synthesis, or combinations of these genetic engineering methods.

The terms “comprising”, “consisting of” and “consisting essentially of” are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated peptides in accordance with the invention preferably do not contain materials normally associated with the peptides in their in situ environment. “Link” or “join” refers to any method known in the art for functionally connecting peptides, including, without limitation, recombinant fusion, covalent bonding, disulfide bonding, ionic bonding, hydrogen bonding, and electrostatic bonding. Additionally, the terms “complementary”, “fully complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” can be used herein as a synonym to “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the specified polynucleotide (e.g., a specified SEQ ID NO:).

The term “fragment(s)”, “probe fragment(s)” or “primer fragment(s)” is used herein to denote a nucleic acid sequence comprising, consisting essentially of, or consisting of at least 8 consecutive nucleotides of any one of SEQ ID NOs: 1, 2, 3, 4, 5, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 108, 110, 111, 112, 113, 114, 115, 116, 117, 118, 119, 120, 121, 122, 123, 124, 125, 126, 127, 128, 129, 120, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212 or 213, said fragment of at least 8 consecutive nucleotides being at least one nucleotide shorter than the number of nucleotides associated with a particular SEQ ID NO: (e.g., any one of SEQ ID NOs: 1-5 and 7-213). The subject invention also provides fragments/primers/probes that comprise, consist essentially of, or consist of 100 or fewer consecutive nucleotides as set forth in SEQ ID NO: 5, 7 or 213, provided that each of said fragments, primers or probes contains a span of at least 8 consecutive nucleotides of at least one sequence as set forth in SEQ ID NOs: 1-4 or 8-212 (or polynucleotide sequences fully complementary thereto). In other words, a fragment, probe, or primer can comprise, consist essentially of, or consist of a contiguous/consecutive span of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 consecutive nucleotides of SEQ ID NO: 5, provided that said contiguous/consecutive span of nucleotides includes at least 8 consecutive nucleotides of at least one of the sequences set forth in SEQ ID NOs: 1, 2, 3 or 4 (or nucleotides sequences fully complementary thereto). In certain embodiments, the primers or probes of SEQ ID NO: 1 comprise, consist essentially of, or consist of at least 8, 9, 10, 11, 12, 13, 14, 15, 16, 17, 18, 19, 20 or 21 consecutive nucleotides as set forth in SEQ ID NO: 1. For SEQ ID NO: 2, various primers or probes according to the subject invention comprise, consist essentially of, or consist of at least 8, 9, 10, 11, 12, 13, 14, 15, 16, 17, 18, 19 or 20 consecutive nucleotides as set forth in SEQ ID NO: 2. With respect to SEQ ID NOs: 3 and 4, primers or probes comprise, consist essentially of, or consist of at least 8, 9, 10, 11, 12, 13, 14, 15, 16, 17 or 18 consecutive nucleotides as set forth in SEQ ID NOs: 3 and 4, respectively. Specifically excluded from the scope of the subject invention is the full length nucleic acid sequence identified in SEQ ID NO: 5, 7 or 213 (accession numbers# U16276, AF286339 and X70277 respectively). The primers, probes, and/or fragments set forth in this paragraph can be, optionally, labeled as set forth below.

The subject invention provides, in one embodiment, methods for the identification of the presence of nucleic acids according to the subject invention in transformed host cells or in cells isolated from an individual suspected of being infected by MAP. In these varied embodiments, the invention provides for the detection of nucleic acids in a sample (obtained from the individual or from a cell culture) comprising contacting a sample with a nucleic acid (polynucleotide) of the subject invention (such as an RNA, mRNA, DNA, cDNA, or other nucleic acid). In a preferred embodiment, the polynucleotide is a probe that is, optionally, labeled and used in the detection system. Many methods for detection of nucleic acids exist and any suitable method for detection is encompassed by the instant invention. Typical assay formats utilizing nucleic acid hybridization includes, and are not limited to, 1) nuclear run-on assay, 2) slot blot assay, 3) northern blot assay (Alwine, et al., Proc. Natl. Acad. Sci. 74:5350), 4) magnetic particle separation, 5) nucleic acid or DNA chips, 6) reverse Northern blot assay, 7) dot blot assay, 8) in situ hybridization, 9) RNase protection assay (Melton, et al., Nuc. Acids Res. 12:7035 and as described in the 1998 catalog of Ambion, Inc., Austin, Tex.), 10) ligase chain reaction, 11) polymerase chain reaction (PCR), 12) reverse transcriptase (RT)-PCR (Berchtold, et al., Nuc. Acids. Res. 17:453), 13) differential display RT-PCR (DDRT-PCR), 14) nested PCR, 15) quantitative PCR or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems. Furthermore, labels useful in producing probes for use in the disclosed methods are well known in the art and widely available to the skilled artisan. Likewise, methods of incorporating labels into the nucleic acids are also well known to the skilled artisan.

Thus, the subject invention also provides detection probes (e.g., fragments of the disclosed polynucleotide sequences) for hybridization with a target sequence or the amplicon generated from the target sequence. Such a fragment or detection probe will comprise a contiguous/consecutive span of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 consecutive nucleotides of SEQ ID NO: 5, provided that said contiguous/consecutive span of nucleotides includes at least 8 consecutive nucleotides of at least one of the sequences set forth in SEQ ID NOs: 1, 2, 3 or 4. Labeled probes or primers can also comprise any one of SEQ ID NOs: 1, 2, 3, 4 or 8-187 or at least 8 consecutive nucleotides of any one of the sequences set forth in SEQ ID NOs: 1, 2, 3, 4 or 8-187. Labeled probes or primers are labeled with a radioactive compound or with another type of label as set forth above (e.g., 1) radioactive labels, 2) enzyme labels, 3) chemiluminescent labels, 4) fluorescent labels, or 5) magnetic labels). Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications.

Polynucleotides of the subject invention can also be used for the qualitative and quantitative analysis of gene expression using arrays or polynucleotides that are attached to a solid support. As used herein, the term array means a one-, two-, or multi-dimensional arrangement of polynucleotides of sufficient length to permit specific detection of gene expression. Preferably, the fragments are at least 15 nucleotides in length and the array contains at least one of SEQ ID NOs: 1, 2, 3, 4, 5, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 108, 110, 111, 112, 113, 114, 115, 116, 117, 118, 119, 120, 121, 122, 123, 124, 125, 126, 127, 128, 129, 120, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, or 186 or any combination thereof (for example, various non-limiting examples are: SEQ ID NO: 1 only, SEQ ID NO: 2 only, SEQ ID NO: 3 only, SEQ ID NO: 4 only, SEQ ID NOs: 1 and 2; SEQ ID NOs: 1 and 3; SEQ ID NOs: 1 and 4; SEQ ID NOs: 2 and 3; SEQ ID NOs: 2 and 4; SEQ ID NOs: 3 and 4 SEQ ID NOs: 1, 2 and 3; SEQ ID NOs: 1, 3 and 4; SEQ ID NOs: 2, 3 and 4; or SEQ ID NOs: 1, 2, 3 and 4). More preferably, the fragments are at least 100 nucleotides in length. More preferably, the fragments are more than 100 nucleotides in length. In some embodiments the fragments may be more than 500 nucleotides in length.

For example, quantitative analysis of gene expression may be performed with full-length polynucleotides of the subject invention, or fragments thereof, in a complementary DNA microarray as described by Schena et al. (Science 270:467-470, 1995; Proc. Natl. Acad. Sci. U.S.A. 93:10614-10619, 1996). Polynucleotides, or fragments thereof, are amplified by PCR and arrayed onto silylated microscope slides. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

mRNA is isolated from a biological sample and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of the polynucleotides present in a biological sample can also be performed in complementary DNA arrays as described by Pietu et al. (Genome Research 6:492-503, 1996). The polynucleotides of the invention, or fragments thereof, are PCR amplified and spotted on membranes. Then, mRNAs originating from biological samples derived from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, the polynucleotide sequences of to the invention may also be used in analytical systems, such as DNA chips. DNA chips and their uses are well known in the art and (see for example, U.S. Pat. Nos. 5,561,071; 5,753,439; 6,214,545; Schena et al., BioEssays, 1996, 18:427-431; Bianchi et al., Clin. Diagn. Virol., 1997, 8:199-208; each of which is hereby incorporated by reference in their entireties) and/or are provided by commercial vendors such as Affymetrix, Inc. (Santa Clara, Calif.). In addition, the nucleic acid sequences of the subject invention can be used as molecular weight markers in nucleic acid analysis procedures.

The phrase “biological sample” is used to denote a sample derived from an individual as defined herein. Such samples include blood samples, serum samples, cellular blood components, milk or other bodily fluids, fecal samples or tissue samples (e.g., tissue biopsies).

The term “individual” is used herein to indicate any non-human animal or human individual that is suspected of being infected by MAP. Thus, various non-limiting examples of “individuals” include apes, chimpanzees, orangutans, humans, monkeys; domesticated animals (pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, giant pandas, hyena, seals, sea lions, and elephant seals. Reptiles include, and are not limited to, alligators, crocodiles, turtles, tortoises, snakes, iguanas, and/or other lizards. Avian species include, and are not limited to, chickens, turkeys, pigeons, quail, parrots, macaws, dove, Guinea hens, lovebirds, parakeets, flamingos, eagles, hawks, falcons, condor, ostriches, peacocks, ducks, and swans.

Prior to conducting an assay for MAP-specific nucleic acids, nucleic acid can be purified from a biological sample if desired. Commercially available kits can be used, according to the manufacturer's recommendations, in the preparation for DNA samples for PCR based methods provided by the subject application. One such kit is the POWERSOIL Soil DNA Extraction Kit (MO BIO Laboratories, Inc., Carlsbad, Calif.). This kit is disclosed in United States Patent Application Publication No. 20050282202A1, Brolaski et al., published Dec. 22, 2005 and in PCT application PCT/US05/17933, Brolaski et al. (PCT publication WO/2006/073472), published Jul. 13, 2006. The disclosure of each of these published applications is hereby incorporated by reference in their entireties and for all purposes. Other methods suitable for purifying nucleic acids from various biological samples can also be used (see, for example, the DNA purification methods discussed in “A rapid, automated protocol for detection of Mycobacterium avian subsp. paratuberculosis in bovine feces and tissues”, Tallec et al., Qiagen News, Issue 6, 2002).

In various aspects of the methods of the invention, the MAP infection can be a subclinical infection, the individual can be a cow or other bovine, and the biological sample can be blood, fecal material or milk. The term “subclinical” is meant as not displaying signs of a disease that are detectable by conventional veterinary or medical examination. In comparison, the term “clinical” means displaying signs of a disease that are detectable by conventional veterinary or medical examination, e.g., rapid weight loss and diarrhea despite good appetite.

In other embodiments, the subject invention provides for diagnostic assays based upon Western blot formats or standard immunoassays known to the skilled artisan that detect antibodies specific for Mycobacterial spp. For example, assays such as enzyme linked immunosorbent assays (ELISAs), radioimmunoassays (RIAs), lateral flow assays, reversible flow chromatographic binding assay (see, for example, U.S. Pat. No. 5,726,010, which is hereby incorporated by reference in its entirety), immunochromatographic strip assays, automated flow assays, and assays utilizing peptide- or antibody-containing biosensors may be employed for the detection of antibodies in the sera of animals/individuals having Johne's Disease.

The assays and methods for conducting the assays are well-known in the art and the methods may test biological samples (e.g., serum, plasma, or blood) qualitatively (presence or absence of antibodies) or quantitatively (comparison of a sample against a standard curve prepared using an antibody standard).

Thus, the subject invention provides a method of identifying animals that have Johne's Disease comprising contacting a test sample with a crude soluble protoplasmic antigen of M. avium detecting the presence of an antibody-antigen complex. A test sample can comprise serum or milk from an individual.

The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are described in: (1) Kusterbeck et al., “Antibody-Based Biosensor for Continuous Monitoring”, in Biosensor Technology, R. P. Buck et al., eds., Marcel Dekker, N.Y. pp. 345-350 (1990); Kusterbeck et al., “A Continuous Flow Immunoassay for Rapid and Sensitive Detection of Small Molecules”, Journal of Immunological Methods, vol. 135, pp. 191-197 (1990); Ligler et al., “Drug Detection Using the Flow Immunosensor”, in Biosensor Design and Application, J. Findley et al., eds., American Chemical Society Press, pp. 73-80 (1992); and Ogert et al., “Detection of Cocaine Using the Flow Immunosensor”, Analytical Letters, vol. 25, pp. 1999-2019 (1992), all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. These methodologies and labels are well known in the art and widely available to the skilled artisan. Likewise, methods of incorporating labels into the nucleic acids are also well known to the skilled artisan. For example, antibodies can be labeled with a radioactive element (³²P, ³⁵S, ³H, ¹²⁵I) or with a molecule such as biotin, acetylaminofluorene, digoxigenin, 5-bromo-deoxyuridine, peroxidase, fluorescein or other labels generally known to the skilled artisan.

Various non-limiting embodiments provided by the subject invention include:

1. A composition of matter comprising:
(a) a PCR primer set specific for Mycobacterium avium subsp. paratuberculosis (MAP) comprising the primers identified in any one of the following primer sets:

| | |
|---|---|
| Primer | |
| Set | SEQ ID NOs: |

| | | |
|---|---|---|
| 1 | 1 | 2 |
| 2 | 3 | 4 |
| 3 | 8 | 9 |
| 4 | 11 | 12 |
| 5 | 14 | 15 |
| 6 | 17 | 18 |
| 7 | 20 | 21 |
| 8 | 23 | 24 |
| 9 | 26 | 27 |
| 10 | 29 | 30 |
| 11 | 32 | 33 |
| 12 | 35 | 36 |
| 13 | 38 | 39 |
| 14 | 41 | 42 |
| 15 | 44 | 45 |
| 16 | 47 | 48 |
| 17 | 50 | 51 |
| 18 | 53 | 54 |
| 19 | 56 | 57 |
| 20 | 59 | 60 |
| 21 | 62 | 63 |
| 22 | 65 | 66 |
| 23 | 68 | 69 |
| 24 | 71 | 72 |
| 25 | 74 | 75 |
| 26 | 77 | 78 |
| 27 | 80 | 81 |
| 28 | 83 | 84 |
| 29 | 86 | 87 |
| 30 | 89 | 90 |
| 31 | 92 | 93 |
| 32 | 95 | 96 |
| 33 | 98 | 99 |
| 34 | 101 | 102 |
| 35 | 104 | 105 |
| 36 | 107 | 108 |
| 37 | 110 | 111 |
| 38 | 113 | 114 |
| 39 | 116 | 117 |
| 40 | 119 | 120 |
| 41 | 122 | 123 |
| 42 | 125 | 126 |
| 43 | 128 | 129 |
| 44 | 131 | 132 |
| 45 | 134 | 135 |
| 46 | 137 | 138 |
| 47 | 140 | 141 |
| 48 | 143 | 144 |
| 49 | 146 | 147 |
| 50 | 149 | 150 |
| 51 | 152 | 153 |
| 52 | 155 | 156 |
| 53 | 158 | 159 |
| 54 | 161 | 162 |
| 55 | 164 | 165 |
| 56 | 167 | 168 |
| 57 | 170 | 171 |
| 58 | 173 | 174 |
| 59 | 176 | 177 |
| 60 | 179 | 180 |
| 61 | 182 | 183 |
| 62 | 185 | 186 |
| 63 | 188 | 189 |
| 64 | 191 | 192 |
| 65 | 194 | 195 |
| 66 | 197 | 198 |
| 67 | 200 | 201 |
| 68 | 203 | 204 |
| 69 | 205 | 206 |
| 70 | 207 | 208 |
| 71 | 209 | 210 |
| 72 | 211 | 212 |

(b) a PCR primer set specific for Mycobacterium avium subsp. paratuberculosis (MAP) comprising the primers identified in any one of the following primer sets:

| Primer | |
|---|---|
| Set | SEQ ID NO: |
| 73 | 8, 9 and 10 |
| 74 | 11, 12 and 13 |
| 75 | 14, 15 and 16 |
| 76 | 17, 18 and 19 |
| 77 | 20, 21 and 22 |
| 78 | 23, 24 and 25 |
| 79 | 26, 27 and 28 |
| 80 | 29, 30 and 31 |
| 81 | 32, 33 and 34 |
| 82 | 35, 36 and 37 |
| 83 | 38, 39 and 40 |
| 84 | 41, 42 and 43 |
| 85 | 44, 45 and 46 |
| 86 | 47, 48 and 49 |
| 87 | 50, 51 and 52 |
| 88 | 53, 54 and 55 |
| 89 | 56, 57 and 58 |
| 90 | 59, 60 and 61 |
| 91 | 62, 63 and 64 |
| 92 | 65, 66 and 67 |
| 93 | 68, 69 and 70 |
| 94 | 71, 72 and 73 |
| 95 | 74, 75 and 76 |
| 96 | 77, 78 and 79 |
| 97 | 80, 81 and 82 |
| 98 | 83, 84 and 85 |
| 99 | 86, 87 and 88 |
| 100 | 89, 90 and 91 |
| 101 | 92, 93 and 94 |
| 102 | 95, 96 and 97 |
| 103 | 98, 99 and 100 |
| 104 | 101, 102 and 103 |
| 105 | 104, 105 and 106 |
| 106 | 107, 108 and 109 |
| 107 | 110, 111 and 112 |
| 108 | 113, 114 and 115 |
| 109 | 116, 117 and 118 |
| 110 | 119, 120 and 121 |
| 111 | 122, 123 and 124 |
| 112 | 125, 126 and 127 |
| 113 | 128, 129 and 130 |
| 114 | 131, 132 and 133 |
| 115 | 134, 135 and 136 |
| 116 | 137, 138 and 139 |
| 117 | 140, 141 and 142 |
| 118 | 143, 144 and 145 |
| 119 | 146, 147 and 148 |
| 120 | 149, 150 and 151 |
| 121 | 152, 153 and 154 |
| 122 | 155, 156 and 157 |
| 123 | 158, 159 and 160 |
| 124 | 161, 162 and 163 |
| 125 | 164, 165 and 166 |
| 126 | 167, 168 and 169 |
| 127 | 170, 171 and 172 |
| 128 | 173, 174 and 175 |
| 129 | 176, 177 and 178 |
| 130 | 179, 180 and 181 |
| 131 | 182, 183 and 184 |
| 132 | 185, 186 and 187 |
| 133 | 188, 189 and 190 |
| 134 | 191, 192 and 193 |
| 135 | 194, 195 and 196 |
| 136 | 197, 198 and 199 |
| 137 | 200, 201 and 202 |

(c) a PCR primer set specific for Mycobacterium avium subsp. paratuberculosis (MAP) comprising the following combinations of primers:

Combinations of Primers (SEQ ID NOs:)

| | | |
|---|---|---|
| 8 and 9 and | 38 and 39 | ; |
| | 41 and 42 | ; |
| | 44 and 45 | ; |
| | 47 and 48 | ; or |
| | 50 and 51 | |
| 11 and 12 and | 53 and 54 | ; |
| | 56 and 57 | ; |
| | 59 and 60 | ; |
| | 62 and 63 | ; or |
| | 65 and 66 | |
| 14 and 15 and | 68 and 69 | ; |
| | 71 and 72 | ; |
| | 74 and 75 | ; |
| | 77 and 78 | ; or |
| | 80 and 81 | |
| 17 and 18 and | 83 and 84 | ; |
| | 86 and 87 | ; |
| | 89 and 90 | ; |
| | 92 and 93 | ; or |
| | 95 and 96 | |
| 20 and 21 and | 98 and 99 | ; |
| | 101 and 102 | ; |
| | 104 and 105 | ; |
| | 107 and 108 | ; or |
| | 110 and 111 | |
| 23 and 24 and | 113 and 114 | ; |
| | 116 and 117 | ; |
| | 119 and 120 | ; |
| | 122 and 123 | ; or |
| | 125 and 126 | |
| 26 and 27 and | 128 and 129 | ; |
| | 131 and 132 | ; |
| | 134 and 135 | ; |
| | 137 and 138 | ; or |
| | 140 and 141 | |
| 29 and 30 and | 143 and 144 | ; |
| | 146 and 147 | ; |
| | 149 and 150 | ; |
| | 152 and 153 | ; or |
| | 155 and 156 | |
| 32 and 33 and | 158 and 159 | ; |
| | 161 and 162 | ; |
| | 164 and 165 | ; |
| | 167 and 168 | ; or |
| | 170 and 171 | |
| 35 and 36 and | 173 and 174 | ; |
| | 176 and 177 | ; |
| | 179 and 180 | ; |
| | 182 and 183 | ; or |
| | 185 and 186 | |
| 188 and 189 and | 203 and 204 | ; |
| | 205 and 206 | ; |
| | 207 and 208 | ; |
| | 209 and 210 | ; or |
| | 211 and 212 | |
| 191 and 192 and | 203 and 204 | ; |
| | 205 and 206 | ; |
| | 207 and 208 | ; |
| | 209 and 210 | ; or |
| | 211 and 212 | |
| 194 and 195 and | 203 and 204 | ; |
| | 205 and 206 | ; |
| | 207 and 208 | ; |
| | 209 and 210 | ; or |
| | 211 and 212 | |
| 197 and 198 and | 203 and 204 | ; |
| | 205 and 206 | ; |
| | 207 and 208 | ; |
| | 209 and 210 | ; or |
| | 211 and 212 | ; or |
| 200 and 201 and | 203 and 204 | ; |
| | 205 and 206 | ; |
| | 207 and 208 | ; |
| | 209 and 210 | ; or |
| | 211 and 212 | |

(d) a PCR primer set specific for Mycobacterium avium subsp. paratuberculosis (MAP) comprising the following combinations of primers:

Combinations of Primers (SEQ ID NOs:)

| | | |
|---|---|---|
| 8 and 9 and 10 and | 38 and 39 | ; |
| | 41 and 42 | ; |
| | 44 and 45 | ; |
| | 47 and 48 | ; or |
| | 50 and 51 | |
| 11 and 12 and 13 | 53 and 54 | ; |
| and | 56 and 57 | ; |
| | 59 and 60 | ; |
| | 62 and 63 | ; or |
| | 65 and 66 | |
| 14 and 15 and 16 | 68 and 69 | ; |
| and | 71 and 72 | ; |
| | 74 and 75 | ; |
| | 77 and 78 | ; or |
| | 80 and 81 | |
| 17 and 18 and 19 | 83 and 84 | ; |
| | 86 and 87 | ; |
| | 89 and 90 | ; |
| | 92 and 93 | ; or |
| | 95 and 96 | |
| 20 and 21 and 22 | 98 and 99 | ; |
| and | 101 and 102 | ; |
| | 104 and 105 | ; |
| | 107 and 108 | ; or |
| | 110 and 111 | |
| 23 and 24 and 25 | 113 and 114 | ; |
| and | 116 and 117 | ; |
| | 119 and 120 | ; |
| | 122 and 123 | ; or |
| | 125 and 126 | |
| 26 and 27 and 28 | 128 and 129 | ; |
| and | 131 and 132 | ; |
| | 134 and 135 | ; |
| | 137 and 138 | ; or |
| | 140 and 141 | |
| 29 and 30 and 31 | 143 and 144 | ; |
| and | 146 and 147 | ; |
| | 149 and 150 | ; |
| | 152 and 153 | ; or |
| | 155 and 156 | |
| 32 and 33 and 34 | 158 and 159 | ; |
| and | 161 and 162 | ; |
| | 164 and 165 | ; |
| | 167 and 168 | ; or |
| | 170 and 171 | |
| 35 and 36 and | 173 and 174 | ; |
| | 176 and 177 | ; |
| | 179 and 180 | ; |
| | 182 and 183 | ; or |
| | 185 and 186 | |
| 188 and 189 and | 203 and 204 | ; |
| 190 and | 205 and 206 | ; |
| | 207 and 208 | ; |
| | 209 and 210 | ; or |
| | 211 and 212 | |
| 191 and 192 and | 203 and 204 | ; |
| 193 and | 205 and 206 | ; |
| | 207 and 208 | ; |
| | 209 and 210 | ; or |
| | 211 and 212 | |
| 194 and 195 and | 203 and 204 | ; |
| 196 and | 205 and 206 | ; |
| | 207 and 208 | ; |
| | 209 and 210 | ; or |
| | 211 and 212 | |
| 197 and 198 and | 203 and 204 | ; |
| 199 and | 205 and 206 | ; |
| | 207 and 208 | ; |
| | 209 and 210 | ; or |
| | 211 and 212 | ; or |
| 200 and 201 and | 203 and 204 | ; |
| 202 and | 205 and 206 | ; |
| | 207 and 208 | ; |
| | 209 and 210 | ; or |
| | 211 and 212 | |

(e) a PCR primer set specific for Mycobacterium avium subsp. paratuberculosis (MAP) comprising the following combinations of primers:

Combinations of Primers
(SEQ ID NOs:)

| | | |
|---|---|---|
| 8 and 9 and | 38 and 39 and 40 | ; or |
| | 41 and 42 and 43 | ; or |
| | 44 and 45 and 46 | ; or |
| | 47 and 48 and 49 | ; or |
| | 50 and 51 and 52 | |
| 11 and 12 | 53 and 54 and 55 | ; or |
| and | 56 and 57 and 58 | ; or |
| | 59 and 60 and 61 | ; or |
| | 62 and 63 and 64 | ; or |
| | 65 and 66 and 67 | |
| 14 and 15 | 68 and 69 and 70 | ; or |
| and | 71 and 72 and 73 | ; or |
| | 74 and 75 and 76 | ; or |
| | 77 and 78 and 79 | ; or |
| | 80 and 81 and 82 | |
| 17 and 18 | 83 and 84 and 85 | ; or |
| and | 86 and 87 and 88 | ; or |
| | 89 and 90 and 91 | ; or |
| | 92 and 93 and 94 | ; or |
| | 95 and 96 and 97 | |
| 20 and 21 | 98 and 99 and 100 | ; or |
| and | 101 and 102 and 103 | ; or |
| | 104 and 105 and 106 | ; or |
| | 107 and 108 and 109 | ; or |
| | 110 and 111 and 112 | |
| 23 and 24 | 113 and 114 and 115 | ; or |
| and | 116 and 117 and 118 | ; or |
| | 119 and 120 and 121 | ; or |
| | 122 and 123 and 124 | ; or |
| | 125 and 126 and 127 | |
| 26 and 27 | 128 and 129 and 130 | ; or |
| and | 131 and 132 and 133 | ; or |
| | 134 and 135 and 136 | ; or |
| | 137 and 138 and 139 | ; or |
| | 140 and 141 and 142 | |
| 29 and 30 | 143 and 144 and 145 | ; or |
| and | 146 and 147 and 148 | ; or |
| | 149 and 150 and 151 | ; or |
| | 152 and 153 and 154 | ; or |
| | 155 and 156 and 157 | |
| 32 and 33 | 158 and 159 and 160 | ; or |
| and | 161 and 162 and 163 | ; or |
| | 164 and 165 and 166 | ; or |
| | 167 and 168 and 169 | ; or |
| | 170 and 171 and 172 | |
| 35 and 36 | 173 and 174 and 175 | ; or |
| and | 176 and 177 and 178 | ; or |
| | 179 and 180 and 181 | ; or |
| | 182 and 183 and 184 | ; or |
| | 185 and 186 and 187 | |

(f) a PCR primer set specific for Mycobacterium avium subsp. paratuberculosis (MAP) comprising the following combinations of primers:

Combinations of Primers
(SEQ ID NOs:)

| | | |
|---|---|---|
| 8 and 9 and | 38 and 39 and 40 | ; or |
| 10 and | 41 and 42 and 43 | ; or |
| | 44 and 45 and 46 | ; or |
| | 47 and 48 and 49 | ; or |
| | 50 and 51 and 52 | |
| 11 and 12 | 53 and 54 and 55 | ; or |
| and 13 and | 56 and 57 and 58 | ; or |
| | 59 and 60 and 61 | ; or |
| | 62 and 63 and 64 | ; or |
| | 65 and 66 and 67 | |
| 14 and 15 | 68 and 69 and 70 | ; or |
| and 16 and | 71 and 72 and 73 | ; or |
| | 74 and 75 and 76 | ; or |
| | 77 and 78 and 79 | ; or |
| | 80 and 81 and 82 | |
| 17 and 18 | 83 and 84 and 85 | ; or |
| and 19 and | 86 and 87 and 88 | ; or |
| | 89 and 90 and 91 | ; or |
| | 92 and 93 and 94 | ; or |
| | 95 and 96 and 97 | |
| 20 and 21 | 98 and 99 and 100 | ; or |
| and 22 and | 101 and 102 and 103 | ; or |
| | 104 and 105 and 106 | ; or |
| | 107 and 108 and 109 | ; or |
| | 110 and 111 and 112 | |
| 23 and 24 | 113 and 114 and 115 | ; or |
| and 25 and | 116 and 117 and 118 | ; or |
| | 119 and 120 and 121 | ; or |
| | 122 and 123 and 124 | ; or |
| | 125 and 126 and 127 | |
| 26 and 27 | 128 and 129 and 130 | ; or |
| and 28 and | 131 and 132 and 133 | ; or |
| | 134 and 135 and 136 | ; or |
| | 137 and 138 and 139 | ; or |
| | 140 and 141 and 142 | |
| 29 and 30 | 143 and 144 and 145 | ; or |
| and 31 and | 146 and 147 and 148 | ; or |
| | 149 and 150 and 151 | ; or |
| | 152 and 153 and 154 | ; or |
| | 155 and 156 and 157 | |
| 32 and 33 | 158 and 159 and 160 | ; or |
| and 34 and | 161 and 162 and 163 | ; or |
| | 164 and 165 and 166 | ; or |
| | 167 and 168 and 169 | ; or |
| | 170 and 171 and 172 | |
| 35 and 36 | 173 and 174 and 175 | ; or |
| and 37 and | 176 and 177 and 178 | ; or |
| | 179 and 180 and 181 | ; or |
| | 182 and 183 and 184 | ; or |
| | 185 and 186 and 187 | |

(g) an isolated polynucleotide comprising any one of SEQ ID NOs: 1 through 212 or an isolated polynucleotide comprising at least 8 consecutive nucleotides of any one of SEQ ID NOs: 1 through 212;
(h) an isolated polynucleotide comprising at least 8 consecutive nucleotides of any one of SEQ ID NOs: 1 through 212, wherein said polynucleotide has a maximum length that is equal to the number of nucleotides associated with said specific SEQ ID NO:;
(i) an isolated polynucleotide that is fully complementary to:
(1) any one of SEQ ID NO: 1 through 212;
(2) a polynucleotide comprising at least 8 consecutive nucleotides of any one of SEQ ID NOs: 1 through 212; or
(3) a polynucleotide comprising at least 8 consecutive nucleotides of any one of SEQ ID NOs: 1 through 212, wherein said polynucleotide has a maximum length that is equal to the number of nucleotides associated with said specific SEQ ID NO:; or
   (j) an isolated polynucleotide comprising a contiguous/consecutive span of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 consecutive nucleotides of SEQ ID NO: 5, 7 or 213 provided that said contiguous/consecutive span of nucleotides includes at least 8 consecutive nucleotides of a primer or probe selected from any one of SEQ ID NOs: 1-4 and 8-212 or polynucleotides fully complementary to any one of SEQ ID NOs: 1-4 and 8-212.
   2. The primer set or isolated polynucleotide according to embodiment 1, wherein one or more of said primers is labeled or said polynucleotide is labeled.
   3. The primer set or isolated polynucleotide according to embodiment 2, wherein said label is a fluorescent label.
   4. The primer set or isolated polynucleotide according to embodiment 2, wherein said label is a radioisotope.
   5. The primer set or isolated polynucleotide according to embodiment 2, wherein said label is biotin.
   6. A method of detecting the presence of Mycobacterium avium subsp. paratuberculosis (MAP) in a sample from individual suspected of being infected with MAP, said method comprising the steps of:
   (a) providing a sample from the individual suspected of being infected with MAP;
   (b) treating the sample to solubilize the nucleic acids therein;
   (c) forming a PCR reaction solution comprising:
   (A) at least a portion of the solubilized nucleic acids from step (b);
   (B) any one of the PCR primer sets according to embodiment 1;
   (C) a mixture of nucleoside triphosphate monomers; and
   (D) a PCR polymerase in a buffered solution;
   (d) carrying out a polymerase chain reaction on the PCR reaction solution to amplify any MAP-specific nucleic acid which is specific for the particular primer set used to a level sufficient for detection; and
   (e) detecting the presence of amplified MAP-specific nucleic acid in the resulting solution which is specific for the particular primer set used; wherein the detection of the amplified MAP-specific nucleic acid which is specific for the particular primer set used indicates that MAP is present in the individual.
   7. The method according to embodiment 6 wherein the sample is a fecal sample from an individual.
   8. The method according to embodiment 7, wherein said individual is a bovine.
   9. The method according to embodiment 6, wherein the primer set comprises primer set 2.
   10. The method according to embodiment 9, wherein the primer set further comprises SEQ ID NO: 1.
   11. The method according to embodiment 9, wherein the primer set further comprises SEQ ID NO: 2.
   12. The method according to embodiment 9, wherein the primer set further comprises SEQ ID NO: 1 and SEQ ID NO: 2.
   13. The method according to embodiment 6, wherein the primer set comprises a polynucleotide comprising at least 8 contiguous nucleotides of SEQ ID NO: 3 and a polynucleotide comprising at least 8 contiguous nucleotides of SEQ ID NO: 4.
   14. The method according to embodiment 13, wherein the primer set further comprises a polynucleotide comprising at least 8 contiguous nucleotides of SEQ ID NO: 1.
   15. The method according to embodiment 13, wherein the primer set further comprises a polynucleotide comprising at least 8 contiguous nucleotides of SEQ ID NO: 2.
   16. The method according to embodiment 13, wherein the primer set further comprises a polynucleotide comprising at least 8 contiguous nucleotides of SEQ ID NO: 1 and a polynucleotide comprising at least 8 contiguous nucleotides of SEQ ID NO: 2.
   17. The method according to embodiment 6, wherein the detection of the presence of amplified MAP-specific nucleic acid comprises gel electrophoresis of the amplified MAP-specific nucleic acid solution and staining of the resulting gel to visualize the band of the MAP-specific nucleic acid specific for the particular primer set used.
   18. The method according to embodiment 17, wherein at least one of the oligonucleotides in the primer set or at least one of the nucleoside triphosphate monomers contains a label which will be incorporated into the amplified MAP-specific nucleic acid and can be used for the detection of the amplified MAP-specific nucleic acid.
   19. A method of detecting the presence of MAP in a sample from individual suspected of being infected with MAP using a nested PCR procedure, said method comprising the steps of:
   (a) providing a sample from the individual suspected of being infected with MAP;
   (b) treating the sample to solubilize the nucleic acids therein;
   (c) forming a first PCR reaction solution containing at least a portion of the solubilized nucleic acids from step (b), a first PCR primer set, a first mixture of nucleoside triphosphate monomers, and a first PCR polymerase in a first buffered solution, wherein the first primer set comprises a first pair of oligonucleotides as set forth in primer set 1, 3, 4, 5, 6, 7, 8, 9, 10, 11, 12, 63, 64, 65, 66 or 67 or fragments of said first pair of oligonucleotides that are at least 8 consecutive nucleotides in length;
   (d) performing a first polymerase chain reaction on the first PCR reaction solution to amplify any MAP-specific nucleic acid which is specific for the first primer set used;
   (e) forming a second PCR reaction solution containing at least a portion of the PCR-reacted first PCR reaction solution from step (d), a second PCR primer set, a second mixture of nucleoside triphosphate monomers, and a second PCR polymerase in a second buffered solution, wherein the second primer set comprises a second pair of oligonucleotides as set forth in primer set 2, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 68, 69, 70, 71 or 72 or fragments of said second pair of oligonucleotides that are at least 8 consecutive nucleotides in length;
   (f) performing a second polymerase chain reaction on the second PCR reaction solution to amplify any MAP-specific nucleic acid which is specific for the second primer set used to a level sufficient for detection; and
   (g) detecting the presence of amplified MAP-specific nucleic acid in the resulting solution from step (f) which specific for the second primer set; wherein the detection of the amplified MAP-specific nucleic acid which is specific for the second primer set indicates that MAP is present in the individual.
   20. The method according to embodiment 19, wherein the sample is a fecal sample from said individual.
   21. The method according to embodiment 20, wherein said individual is a bovine.
   22. The method according to embodiment 19, wherein the detection in step (g) comprises gel electrophoresis of the amplified MAP-specific nucleic acid solution and staining of the resulting gel to visualize the MAP-specific nucleic acid on the gel.
   23. The method according to embodiment 19, wherein either the primers, or one or more of the monomers, or both, employed in steps (c) and (e) contains a label whereby the amplified MAP-specific nucleic acid that results in step (f) contains the label, and the detection in step (g) comprises detecting the presence of the label.
   24. The method according to embodiment 19, wherein said first primer set comprises the oligonucleotides of primer set 1 (SEQ ID NO: 1 and 2) or fragments of SEQ ID NO: 1 and SEQ ID NO:2 that comprise at least 8 contiguous nucleotides of SEQ ID NOs: 1 and 2.
   25. The method according to embodiment 19, wherein said second primer set comprises the oligonucleotides of primer set 2 (SEQ ID NOs: 3 and 4) or fragments of SEQ ID NO:3 and SEQ ID NO:4 that comprise at least 8 contiguous nucleotides of SEQ ID NOs:3 and 4.
   26. The method according to embodiment 19, wherein said first primer set comprises fragments of at least 8 consecutive nucleotides of SEQ ID NOs:1 and 2 and said second primer set comprises fragments of at least 8 consecutive nucleotides of SEQ ID NOs:3 and 4.
   27. An improvement in a PCR-based method of detecting the presence of Mycobacterium avium subsp. paratuberculosis (MAP) in a sample from individual suspected of being infected with MAP, wherein the improvement comprises the use of a polynucleotide or primer set as set forth in embodiment 1.
   28. A method of identifying animals having Johne's Disease comprising:
   a) obtaining sera from an animal suspected of having Johne's disease;
   b) contacting a crude soluble protoplasmic antigen of M. avium with sera from said animal (test sera) and a control sera; and
   c) detecting the binding of antibodies to said crude protoplasmic antigen, wherein an animal having Johne's disease is identified when the amount of test sera antibody bound to the crude soluble antigen is greater than the amount of a control sera antibody bound to said crude soluble antigen.
   29. The method according to embodiment 27, wherein the sera obtained from said animal has been preabsorbed with Mycobacterium pheli.
   30. The method according to embodiment 27 or 28, wherein said detecting comprises contacting the antibodies of said test sera and said control sera with a labeled antibody.
   31. The method according to embodiment 29, wherein said antibody is labeled with an fluorophore, an enzyme, or a radiolabel.
   32. The method according to any one of embodiments 6 through 27, further comprising the detection of amplified gene product with a probe.
   33. The method according to embodiment 32, wherein said probe comprises a label that is a fluorescent dye or radiolabel.
   34. The method according to embodiment 33, wherein said probe comprises a fluorescent dye and a quencher.
   35. The method according to embodiment 34, wherein probe is 5′-/56-FAM/CAC ACT GTC GAC GAT CGC/31ABlkFQ/-3′.

Primers and combinations of primers that are suitable for use in the practice of the PCR based methods set forth herein are the various oligonucleotides identified as a “primer” in the tables that are set forth below.

| | | | Possible |
|---|---|---|---|
| | | SEQ | Member |
| | | ID | of Primer |
| | Sequence | NO: | Set No. |
| F Primers | | | |
| Primer F1 | gtcattcagaatcgctgcaa | 8 | 3 or 73 |
| Primer F2 | tggcgtcagctattggtgta | 9 | 3 or 73 |
| Probe F1F2 | aactcgaacacacctgggac | 10 | 3 or 73 |
| Primer F3 | tcctctccttcgtcaccaac | 11 | 4 or 74 |
| Primer F4 | atgaaatgggcgtctaccag | 12 | 4 or 74 |
| Probe F3F4 | gtcattcagaatcgctgcaa | 3 | 4 or 74 |
| Primer F5 | gtcattcagaatcgctgcaa | 14 | 5 or 75 |
| Primer F6 | cgtcagctattggtgtaccg | 15 | 5 or 75 |
| Probe F5F6 | aactcgaacacacctgggac | 16 | 5 or 75 |
| Primer F7 | cattcagaatcgctgcaatc | 17 | 6 or 76 |
| Primer F8 | tggcgtcagctattggtgta | 18 | 6 or 76 |
| Probe F7F8 | aactcgaacacacctgggac | 19 | 6 or 76 |
| Primer F9 | agaatcgctgcaatctcagg | 20 | 7 or 77 |
| Primer F10 | tggcgtcagctattggtgta | 21 | 7 or 77 |
| Probe F9F10 | aactcgaacacacctgggac | 22 | 7 or 77 |
| M primers | | | |
| Primer M1 | cgaatcgcgttacatcacag | 23 | 8 or 78 |
| Primer M2 | gaaaccacgttgcgagtacc | 24 | 8 or 78 |
| Probe M1M2 | taccgactgagctacctggc | 25 | 8 or 78 |
| Primer M3 | atcacaggtcttccggteae | 26 | 9 or 79 |
| Primer M4 | gaaaccacgttgcgagtacc | 27 | 9 or 79 |
| Probe M3M4 | taccgactgagctacctggc | 28 | 9 or 79 |
| Primer M5 | gacgaatcgcgttacatcac | 29 | 10 or 80 |
| Primer M6 | gaaaccacgttgcgagtacc | 30 | 10 or 80 |
| Probe M5M6 | taccgactgagctacctggc | 31 | 10 or 80 |
| Primer M7 | tcgcgttacatcacaggtct | 32 | 11 or 81 |
| Primer M8 | gaaaccacgttgcgagtacc | 33 | 11 or 81 |
| Probe M7M8 | taccgactgagctacctggc | 34 | 11 or 81 |
| Primer M9 | gaatcgcgttacatcacagg | 35 | 12 or 82 |
| Primer M10 | gaaaccacgttgcgagtacc | 36 | 12 or 82 |
| Probe M9M10 | taccgactgagctacctggc | 37 | 12 or 82 |
| Nested Primers | | | |
| for amplicon | | | |
| produced by F1 | | | |
| and F2 | | | |
| Primer F1F2N1 | gtcattcagaatcgctgcaa | 38 | 13 or 83 |
| Primer F1F2N2 | cgtggtctctgagtttgggta | 39 | 13 or 83 |
| Probe | ctggtagacgcccatttcat | 40 | 13 or 83 |
| F1F2N1F1F2N2 | | | |
| Primer F1F2N3 | gtcattcagaatcgctgcaa | 41 | 14 or 84 |
| Primer F1F2N4 | tatcgatgaaatgggcgtct | 42 | 14 or 84 |
| Probe | cagctccagatcgtcattca | 43 | 14 or 84 |
| F1F2N3F1F2N4 | | | |
| Primer F1F2N5 | gtcattcagaatcgctgcaa | 44 | 15 or 85 |
| Primer F1F2N6 | ccactcgtggtctctgagttt | 45 | 15 or 85 |
| Probe | ctggtagacgcccatttcat | 46 | 15 or 85 |
| F1F2N5F1F2N6 | | | |
| Primer F1F2N7 | gtcattcagaatcgctgcaa | 47 | 16 or 86 |
| Primer F1F2N8 | atcgatgaaatgggcgtcta | 48 | 16 or 86 |
| Probe | cagctccagatcgtcattca | 49 | 16 or 86 |
| F1F2N7F1F2N8 | | | |
| Primer F1F2N9 | gtcattcagaatcgctgcaa | 50 | 17 or 87 |
| Primer F1F2N10 | ctcgtggtctctgagtttgg | 51 | 17 or 87 |
| Probe | ctggtagacgcccatttcat | 52 | 17 or 87 |
| F1F2N9F1F2N10 | | | |
| Nested Primers | | | |
| for amplicon | | | |
| produced by F3 | | | |
| and F4 | | | |
| Primer F3F4N1 | gtcattcagaatcgctgcaa | 53 | 18 or 88 |
| Primer F3F4N2 | cgtggtctctgagtttgggta | 54 | 18 or 88 |
| Probe | ctggtagacgcccatttcat | 55 | 18 or 88 |
| F3F4N1F3F4N2 | | | |
| Primer F3F4N3 | gtcattcagaatcgctgcaa | 56 | 19 or 89 |
| Primer F3F4N4 | tatcgatgaaatgggcgtct | 57 | 19 or 89 |
| Probe | cagctccagatcgtcattca | 58 | 19 or 89 |
| F3F4N3F3F4N4 | | | |
| Primer F3F4N5 | gtcattcagaatcgctgcaa | 59 | 20 or 90 |
| Primer F3F4N6 | ccactcgtggtctctgagttt | 60 | 20 or 90 |
| Probe | ctggtagacgcccatttcat | 61 | 20 or 90 |
| F3F4N5F3F4N6 | | | |
| Primer F3F4N7 | gtcattcagaatcgctgcaa | 62 | 21 or 91 |
| Primer F3F4N8 | atcgatgaaatgggcgtcta | 63 | 21 or 91 |
| Probe | cagctccagatcgtcattca | 64 | 21 or 91 |
| F3F4N7F3F4N8 | | | |
| Primer F3F4N9 | gtcattcagaatcgctgcaa | 65 | 22 or 92 |
| Primer F3F4N10 | ctcgtggtctctgagtttgg | 66 | 22 or 92 |
| Probe | ctggtagacgcccatttcat | 67 | 22 or 92 |
| F3F4N9F3F4N10 | | | |
| Nested Primers | | | |
| for amplicon | | | |
| produced by F5 | | | |
| and F6 | | | |
| Primer F5F6N1 | agaatcgctgcaatctcagg | 68 | 23 or 93 |
| Primer F5F6N2 | cgtggtctctgagtttgggta | 69 | 23 or 93 |
| Probe | cgcttgaatggtcgtctgt | 70 | 23 or 93 |
| F4F6N1F5F6N2 | | | |
| Primer F5F6N3 | agaatcgctgcaatctcagg | 71 | 24 or 94 |
| Primer F5F6N4 | cttagttcgccgcttgaatg | 72 | 24 or 94 |
| Probe | ctggtagacgcccatttcat | 73 | 24 or 94 |
| F5F6N3F5F6N4 | | | |
| Primer F5F6N5 | agaatcgctgcaatctcagg | 74 | 25 or 95 |
| Primer F5F6N6 | ccactcgtggtctctgagttt | 75 | 25 or 95 |
| Probe | ctggtagacgcccatttcat | 76 | 25 or 95 |
| F5F6N5F5F6N6 | | | |
| Primer F5F6N7 | ctgcaatctcaggcagctc | 77 | 26 or 96 |
| Primer F5F6N8 | cttagttcgccgcttgaatg | 78 | 26 or 96 |
| Probe | ctggtagacgcccatttcat | 79 | 26 or 96 |
| F5F6N7F5F6N8 | | | |
| Primer F5F6N9 | ctgcaatctcaggcagctc | 80 | 27 or 97 |
| Primer F5F6N10 | ttagttcgccgcttgaatg | 81 | 27 or 97 |
| Probe | ctggtagacgcccatttcat | 82 | 27 or 97 |
| F5F6N9F5F6N10 | | | |
| Nested Primers | | | |
| for amplicon | | | |
| produced by F7 | | | |
| and F8 | | | |
| Primer F7F8N1 | cagctccagatcgtcattca | 83 | 28 or 98 |
| Primer F7F8N2 | tgtcgatccgcttagttcg | 84 | 28 or 98 |
| Probe | ctggtagacgcccatttcat | 85 | 28 or 98 |
| F7F8N1F7F8N2 | | | |
| Primer F7F8N3 | gcattccaagtcctgaccac | 86 | 29 or 99 |
| Primer F7F8N4 | gtcccaggtgtgttcgagtt | 87 | 29 or 99 |
| Probe | ctggtagacgcccatttcat | 88 | 29 or 99 |
| F7F8N3F7F8N4 | | | |
| Primer F7F8N5 | cagctccagatcgtcattca | 89 | 30 or 100 |
| Primer F7F8N6 | ttgtcgatccgcttagttcg | 90 | 30 or 100 |
| Probe | ctggtagacgcccatttcat | 91 | 30 or 100 |
| F7F8N5F7F8N6 | | | |
| Primer F7F8N7 | agaatcgctgcaatctcagg | 92 | 31 or 101 |
| Primer F7F8N8 | cgcttgaatggtcgtctgt | 93 | 31 or 101 |
| Probe | ctggtagacgcccatttcat | 94 | 31 or 101 |
| F7F8N7F7F8N8 | | | |
| Primer F7F8N9 | agaatcgctgcaatctcagg | 95 | 32 or 102 |
| Primer F7F8N10 | cttagttcgccgcttgaatg | 96 | 32 or 102 |
| Probe | ctggtagacgcccatttcat | 97 | 32 or 102 |
| F7F8N9F7F8N10 | | | |
| Nested Primers | | | |
| for amplicon | | | |
| produced by F9 | | | |
| and F10 | | | |
| Primer F9F10N1 | cagctccagatcgtcattca | 98 | 33 or 103 |
| Primer F9F10N2 | tgtcgatccgcttagttcg | 99 | 33 or 103 |
| Probe | ctggtagacgcccatttcat | 100 | 33 or 103 |
| F9F10N1F9F10N2 | | | |
| Primer F9F10N3 | cagctccagatcgtcattca | 101 | 34 or 104 |
| Primer F9F10N4 | ttgtcgatccgcttagttcg | 102 | 34 or 104 |
| Probe | ctggtagacgcccatttcat | 103 | 34 or 104 |
| F9F10N3F9F10N4 | | | |
| Primer F9F10N5 | gcattccaagtcctgaccac | 104 | 35 or 105 |
| Primer F9F10N6 | caggtgtgttcgagttgcag | 105 | 35 or 105 |
| Probe | ctggtagacgcccatttcat | 106 | 35 or 105 |
| F9F10N5F9F10N6 | | | |
| Primer F9F10N7 | gcagctccagatcgtcattc | 107 | 36 or 106 |
| Primer F9F10N8 | tgtcgatccgcttagttcg | 108 | 36 or 106 |
| Probe | ctggtagacgcccatttcat | 109 | 36 or 106 |
| F9F10N7F9F10N8 | | | |
| Primer F9F10N9 | cagctccagatcgtcattca | 110 | 37 or 107 |
| Primer E9F10N10 | tgagaattgtcgatccgctta | 111 | 37 or 107 |
| Probe | ctggtagacgcccatttcat | 112 | 37 or 107 |
| F9F10N9F9F10N10 | | | |
| Nested Primers | | | |
| for amplicon | | | |
| produced by M1 | | | |
| and M2 | | | |
| Primer M1M2N1 | ggcagcatgctcaagtagc | 113 | 38 or 108 |
| Primer M1M2N2 | gggttcgaatcccgtagg | 114 | 38 or 108 |
| Probe | taccgactgagctacctggc | 115 | 38 or 108 |
| M1M2N1M1M2N2 | | | |
| Primer M1M2N3 | gcagcatgctcaagtagcc | 116 | 39 or 109 |
| Primer M1M2N4 | gggttcgaatcccgtagg | 117 | 39 or 109 |
| Probe | taccgactgagctacctggc | 118 | 39 or 109 |
| M1M2N3M1M2N4 | | | |
| Primer M1M2N5 | gcagcatgctcaagtagcc | 119 | 40 or 110 |
| Primer M1M2N6 | ccctttcaaggcggtagc | 120 | 40 or 110 |
| Probe | taccgactgagctacctggc | 121 | 40 or 110 |
| M1M2N5M1M2N6 | | | |
| Primer M1M2N7 | gcagcatgctcaagtagcc | 122 | 41 or 111 |
| Primer M1M2N8 | gccctttcaaggcggtag | 123 | 41 or 111 |
| Probe | taccgactgagctacctggc | 124 | 41 or 111 |
| M1M2N7M1M2N8 | | | |
| Primer M1M2N9 | ggcagcatgctcaagtagc | 125 | 42 or 112 |
| Primer M1M2N10 | ccctttcaaggcggtagc | 126 | 42 or 112 |
| Probe | taccgactgagctacctggc | 127 | 42 or 112 |
| M1M2N9M1M2N10 | | | |
| Nested Primers | | | |
| for amplicon | | | |
| produced by M3 | | | |
| and M4 | | | |
| Primer M3M4N1 | ggcagcatgctcaagtagc | 128 | 43 or 113 |
| Primer M3M4N2 | gggttcgaatcccgtagg | 129 | 43 or 113 |
| Probe | taccgactgagctacctggc | 130 | 43 or 113 |
| M3M4N1M3M4N2 | | | |
| Primer M3M4N3 | gcagcatgctcaagtagcc | 131 | 44 or 114 |
| Primer M3M4N4 | gggttcgaatcccgtagg | 132 | 44 or 114 |
| Probe | taccgactgagctacctggc | 133 | 44 or 114 |
| M3M4N3M3M4N4 | | | |
| Primer M3M4N5 | gcagcatgctcaagtagcc | 134 | 45 or 115 |
| Primer M3M4N6 | ccctttcaaggcggtagc | 135 | 45 or 115 |
| Probe | taccgactgagctacctggc | 136 | 45 or 115 |
| M3M4N5M3M4N6 | | | |
| Primer M3M4N7 | gcagcatgctcaagtagcc | 137 | 46 or 116 |
| Primer M3M4N8 | gccctttcaaggcggtag | 138 | 46 or 116 |
| Probe | taccgactgagctacctggc | 139 | 46 or 116 |
| M3M4N7M3M4N8 | | | |
| Primer M3M4N9 | ggcagcatgctcaagtagc | 140 | 47 or 117 |
| Primer M3M4N10 | ccctttcaaggcggtagc | 141 | 47 or 117 |
| Probe | taccgactgagctacctggc | 142 | 47 or 117 |
| M3M4N9M3M4N10 | | | |
| Nested Primers | | | |
| for amplicon | | | |
| produced by M5 | | | |
| and M6 | | | |
| Primer M5M6N1 | ggcagcatgctcaagtagc | 143 | 48 or 118 |
| Primer M5M6N2 | ctgtggcgcagttggttag | 144 | 48 or 118 |
| Probe | taccgactgagctacctggc | 145 | 48 or 118 |
| M5M6N1M5M6N2 | | | |
| Primer M5M6N3 | gcagcatgctcaagtagcc | 146 | 49 or 119 |
| Primer M5M6N4 | ctgtggcgcagttggttag | 147 | 49 or 119 |
| Probe | taccgactgagctacctggc | 148 | 49 or 119 |
| M5M6N3M5M6N4 | | | |
| Primer M5M6N5 | cggcagcatgctcaagtag | 149 | 50 or 120 |
| Primer M5M6N6 | ctgtggcgcagttggttag | 150 | 50 or 120 |
| Probe | taccgactgagctacctggc | 151 | 50 or 120 |
| M5M6N5M5M6N6 | | | |
| Primer M5M6N7 | cggcagcatgctcaagta | 152 | 51 or 121 |
| Primer M5M6N8 | ctgtggcgcagttggttag | 153 | 51 or 121 |
| Probe | taccgactgagctacctggc | 154 | 51 or 121 |
| M5M6N7M5M6N8 | | | |
| Primer M5M6N9 | ggcagcatgctcaagtagc | 155 | 52 or 122 |
| Primer M5M6N10 | gtggcgcagttggttagc | 156 | 52 or 122 |
| Probe | taccgactgagctacctggc | 157 | 52 or 122 |
| M5M6N9M5M6N10 | | | |
| Nested Primers | | | |
| for amplicon | | | |
| produced by M7 | | | |
| and M8 | | | |
| Primer M7M8N1 | ggcagcatgctcaagtagc | 158 | 53 or 123 |
| Primer M7M8N2 | gggttcgaatcccgtagg | 159 | 53 or 123 |
| Probe | taccgactgagctacctggc | 160 | 53 or 123 |
| M7M8N1M7M8N2 | | | |
| Primer M7M8N3 | gcagcatgctcaagtagcc | 161 | 54 or 124 |
| Primer M7M8N4 | gggttcgaatcccgtagg | 162 | 54 or 124 |
| Probe | taccgactgagctacctggc | 163 | 54 or 124 |
| M7M8N3M7M8N4 | | | |
| Primer M7M8N5 | gcagcatgctcaagtagcc | 164 | 55 or 125 |
| Primer M7M8N6 | ccctttcaaggcggtagc | 165 | 55 or 125 |
| Probe | taccgactgagctacctggc | 166 | 55 or 125 |
| M7M8N5M7M8N6 | | | |
| Primer M7M8N7 | gcagcatgctcaagtagcc | 167 | 56 or 126 |
| Primer M7M8N8 | gccctttcaaggcggtag | 168 | 56 or 126 |
| Probe | taccgactgagctacctggc | 169 | 56 or 126 |
| M7M8N7M7M8N8 | | | |
| Primer M7M8N9 | ggcagcatgctcaagtagc | 170 | 57 or 127 |
| Primer M7M8N10 | ccctttcaaggcggtagc | 171 | 57 or 127 |
| Probe | taccgactgagctacctggc | 172 | 57 or 127 |
| M7M8N9M7M8N10 | | | |
| Nested Primers | | | |
| for amplicon | | | |
| produced by M9 | | | |
| and M10 | | | |
| Primer M9M10N1 | gcagcatgctcaagtagcc | 173 | 58 or 128 |
| Primer M9M10N2 | aatcccgtagggggtacg | 174 | 58 or 128 |
| Probe | taccgactgagctacctggc | 175 | 58 or 128 |
| M9M10N1M9M10N2 | | | |
| Primer M9M10N3 | ggcagcatgctcaagtagc | 176 | 59 or 129 |
| Primer M9M10N4 | aatcccgtagggggtacg | 177 | 59 or 129 |
| Probe | taccgactgagctacctggc | 178 | 59 or 129 |
| M9M10N3M9M10N4 | | | |
| Primer M9M10N5 | gcagcatgctcaagtagcc | 179 | 60 or 130 |
| Primer M9M10N6 | gaatcccgtagggggtacg | 180 | 60 or 130 |
| Probe | taccgactgagctacctggc | 181 | 60 or 130 |
| M9M10N5M9M10N6 | | | |
| Primer M9M10N7 | ggcagcatgctcaagtagc | 182 | 61 or 131 |
| Primer M9M10N8 | gaatcccgtagggggtacg | 183 | 61 or 131 |
| Probe | taccgactgagctacctggc | 184 | 61 or 131 |
| M9M10N7M9M10N8 | | | |
| Primer M9M10N9 | gcagcatgctcaagtagcc | 185 | 62 or 132 |
| Primer M9M10N10 | gggttcgaatcccgtagg | 186 | 62 or 132 |
| Probe | taccgactgagctacctggc | 187 | 62 or 132 |
| M9M10N9M9M10N10 | | | |
| P900 Series | | | |
| Primers | | | |
| Primer P901 | ggcacggctcttgttgtagt | 188 | 63 or 133 |
| Primer P902 | gcgctgctggagttgatt | 189 | 63 or 133 |
| Probe P901P902 | gaatataaagcagccgctgc | 190 | 63 or 133 |
| Primer P901A | cacggctcttgttgtagtcg | 191 | 64 or 134 |
| Primer P902A | gcgctgctggagttgatt | 192 | 64 or 134 |
| Probe P901AP902A | gaatataaagcagccgctgc | 193 | 64 or 134 |
| Primer P901B | cggctcttgttgtagtcgaa | 194 | 65 or 135 |
| Primer P902B | gcgctgctggagttgatt | 195 | 65 or 135 |
| Probe P901BP902B | gaatataaagcagccgctgc | 196 | 65 or 135 |
| Primer P901C | cggctcttgttgtagtcgaag | 197 | 66 or 136 |
| Primer P902C | gcgctgctggagttgatt | 198 | 66 or 136 |
| Probe P901CP902C | gaatataaagcagccgctgc | 199 | 66 or 136 |
| Primer P901D | acggctcttgttgtagtcgaa | 200 | 67 or 137 |
| Primer P902D | gcgctgctggagttgatt | 201 | 67 or 137 |
| Probe P901DP902D | gaatataaagcagccgctgc | 202 | 67 or 137 |
| Nested Primers | | | |
| for amplicon | | | |
| produced by P901 | | | |
| and 902 | | | |
| Series Primers | | | |
| Primer P901N | gttccagcgccgaaagtat | 203 | 63, 64, |
| | | | 65, 66, |
| | | | 67 or 68 |
| Primer P902N | caagaccgacgccaaagac | 204 | 63, 64, |
| | | | 65, 66, |
| | | | 67 or 68 |
| Primer P901AN | gttccagcgccgaaagtat | 205 | 63, 64, |
| | | | 65, 66, |
| | | | 67 or 69 |
| Primer P902AN | caagaccgacgccaaaga | 206 | 63, 64, |
| | | | 65, 66, |
| | | | 67 or 69 |
| Primer P901BN | gttccagcgccgaaagtatt | 207 | 63, 64, |
| | | | 65, 66, |
| | | | 67 or 70 |
| Primer P902BN | caagaccgacgccaaagac | 208 | 63, 64, |
| | | | 65, 66, |
| | | | 67 or 70 |
| Primer P901CN | agcgccgaaagtattccag | 209 | 63, 64, |
| | | | 65, 66, |
| | | | 67 or 71 |
| Primer P902CN | caagaccgacgccaaagac | 210 | 63, 64, |
| | | | 65, 66, |
| | | | 67 or 71 |
| Primer P901DN | gttccagcgccgaaagtatt | 211 | 63, 64, |
| | | | 65, 66, |
| | | | 67 or 72 |
| Primer P902DN | caagaccgacgccaaaga | 212 | 63, 64, |
| | | | 65, 66, |
| | | | 67 or 72 |

With respect to various nested PCR techniques for which the primers of the subject invention are useful, various combinations of “appropriate” primer sets are set forth in the following table. Primer sets identified as “Appropriate Second PCR Primer Sets” can be used to amplify the amplicon generated by the “First PCT Primer Set”.

| | Appropriate Second |
|---|---|
| First PCR Primer Set | PCR Primer Sets |
| 1 | 2 |
| 3 | 13, 14, 15, 16 or 17 |
| 4 | 18, 19, 20, 21 or 22 |
| 5 | 23, 24, 25, 26 or 27 |
| 6 | 28, 29, 30, 31 or 32 |
| 7 | 33, 34, 35, 36 or 37 |
| 8 | 38, 39, 40, 41 or 42 |
| 9 | 43, 44, 45 46 or 47 |
| 10 | 48, 49, 50, 51 or 52 |
| 11 | 53, 54, 55, 56 or 57 |
| 12 | 58, 59, 60, 61 or 61 |
| 63 or 64 or 65 or 66 or 67 | 68 or 69 or 70 or 71 or 72 |

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### Example 1

### Materials and Methods

### Sample Handling and Nested PCR Protocol

Samples may have the consistency of wet grass to a sticky paste that can be molded, to a semi-liquid, making it a challenge to weigh efficiently. Samples that are liquid to a semi-liquid can be measured using disposable transfer pipette. For really viscous samples, cut the tip from the pipette to increase the diameter of the bore and this will aid in sampling. When using a pipette to measure, it is preferable to use between 250 and 300 ul of sample, to avoid over-load of the bead sample tube.

### Performing PCR (Amplifying the IS1311 Sequence)

| | | |
|---|---|---|
| Master Mix: | 20 ul | Master Mix (supplied with kit) |
| | 19 ul | PCR Quality Water (supplied with kit) |
| | 0.5 ul | Primer #1 (supplied with kit) |
| | 0.5 ul | Primer #2 (supplied with kit) |
| | 40.0 ul | |
| | 10.0 ul | Processed fecal sample |
| | 50.0 ul | |

The following primers were used for Standard PCR:

| | |
|---|---|
| IS1 | |
| 5′-CGA TTT ATC AGG CAC TCA TCG -3′ | (SEQ ID NO: 1) |
| IS2 | |
| 5′-CAA ATA GGC CTC CAT CAC CA-3′ | (SEQ ID NO: 2) |

IS2 & IS2 produce a product of 242 base pairs

### Amplifications: Standard PCR

2 min@94C
30 cycles of: 30 sec@94C
15 sec@58C
60 sec@72C

### Hold@4C

The following primers were used for Nested PCR:

| | | |
|---|---|---|
| IS3 | 5′-ATG AAC GGA GCG CAT CAC-3′ | (SEQ ID NO: 3) |
| IS4 | 5′-CGA CCG AAG CTT GGG AAT-3′ | (SEQ ID NO: 4) |

IS3 & IS4 produce a product of 104 base pairs

### Amplifications: Nested PCR

Master Mix is the same as Standard PCR with the exception that the volume of water is increased from 19 ul to 28 ul and a 1.0 ul sample of the Standard PCR reaction is used instead of 10 ul as in the fecal processing sample.
2 min@94C
30 cycles of: 30 sec@94C
15 sec@63C
60 sec@72C

### Hold@4C

Samples from USDA Johne's Fecal Check Test (KIT #105 from USDA)

Using PowerSoil DNA Kit (MO BIO) previous to PCR

| | USDA | Colonies/ | | | | |
|---|---|---|---|---|---|---|
| USDA # | key | tube | P90-P91 | J1-J2 | IS1-IS2 | IS3-IS4 |
| 1 | + | 15 | − | + | − | + |
| 2 | + | TNTC | + | + | + | + |
| 3 | − | 0 | − | − | − | − |
| 4 | + | ? | + | + | + | + |
| 5 | − | 0 | − | − | − | − |
| 6 | + | 9 | − | + | − | + |
| 7 | − | 0 | − | − | − | − |
| 8 | + | TNTC | − | + | − | + |
| 9 | + | 5 | − | + | − | + |
| 10 | + | TNTC | − | + | − | + |
| 11 | + | 4 | − | + | − | + |
| 12 | + | 14 | + | + | + | + |
| 13 | − | 0 | − | − | − | − |
| 14 | + | TNTC | + | + | − | + |
| 15 | + | 1 | + | + | + | + |
| 16 | + | 15 | + | + | + | + |
| 17 | − | 0 | − | − | − | − |
| 18 | + | 1 | + | + | + | + |
| 19 | + | 1 | + | + | + | + |
| 20 | − | 0 | − | − | − | − |
| 21 | + | 1 | + | + | + | + |
| 22 | + | 6 | − | + | − | + |
| 23 | + | 9 | + | + | + | + |
| 24 | − | 0 | − | − | − | − |
| 25 | + | 1 | + | + | + | + |
| 26 | + | TNTC | + | + | + | + |

P90-P91 flanking primers for IS900;
J1-J2 nested PCR primers for P90-P91 amplicon
IS1-IS2 flanking primers for IS1311;
IS3-IS4 nested PCR primers for IS1-IS2 amplicon

Twenty six (26) fecal samples were provided by APHIS/USDA with known MAP infection status. However, the status (+/−) of these blinded samples was unknown until after results of the PCR assays were communicated to USDA. As indicated in the Table, the IS3 and IS4 primers identified each of the samples known to be derived from MAP infected animals. Based on these results, the laboratory met the qualification requirements of the USDA as a diagnostic center for MAP.

Samples from USDA Johne's Fecal Check Test (Kit #F1)

Using DNA purification Kit (patent pending) previous to PCR

| USDA | USDA | P90- | | P901- | | | |
|---|---|---|---|---|---|---|---|
| # | Key | P91 | J1-J2 | P902 | P903-P904 | IS1-IS2 | IS3-IS4 |
| 1 | − | − | − | − | − | − | − |
| 2 | + | − | + | − | + | − | + |
| 3 | + | − | + | − | + | − | + |
| 4 | + | − | − | − | − | − | − |
| 5 | + | − | + | − | + | − | + |
| 6 | + | − | + | − | + | − | + |
| 7 | + | − | − | − | + | − | + |
| 8 | − | − | − | − | − | − | − |
| 9 | + | − | + | − | + | − | + |
| 10 | + | − | + | − | + | − | + |
| 11 | − | − | − | − | − | − | − |
| 12 | + | − | + | − | + | − | + |
| 13 | − | − | − | − | − | − | − |
| 14 | + | − | + | − | + | − | + |
| 15 | + | − | + | − | + | − | + |
| 16 | + | − | + | − | + | − | + |
| 17 | + | − | + | − | + | − | + |
| 18 | − | − | − | − | − | − | − |
| 19 | + | − | + | + | + | − | + |
| 20 | + | − | + | + | + | − | + |
| 21 | − | − | − | − | − | − | − |
| 22 | + | − | + | − | + | − | + |
| 23 | + | − | − | − | − | − | − |
| 24 | − | − | − | − | − | − | − |
| 25 | + | − | + | − | + | − | + |

Fecal samples were provided by USDA with known MAP infection status. However, the status (+/−) of these samples was unknown until after results of the PCR assays had been communicated to USDA. As indicated in the Table, the IS3 and IS4 primers, the P902 and P903 primers and the J1 and J2 primers identified each of the samples known to be derived from MAP infected animals.

Statistical comparison of P90-P91/J1-J2 versus IS1-IS2/IS3-IS4 primers
on three USDA laboratory certification kit fecal specimens

| | P90-P91/J1-J2 | IS1-IS2/IS3-IS4 |
|---|---|---|
| Sensitivity | 89.3% | 96.5% |
| Specificity | 90.5% | 95.2% |
| Kappa Coefficient | 0.753 | 0.903 |
| Interpretation | Good agreement | Very good agreement |

Comparison of primers P90-P91, IS1-IS2, P90-P91/J1-J2
and IS1-IS2/IS3-IS4 false positive and false negative observed
on three USDA certification kit fecal specimens
Primers

| | P90-P91 | IS1-IS2 | P90-P91/J1-J2 | IS1-IS2/IS3-IS4 |
|---|---|---|---|---|
| False Positive | 20 | 26 | 2 | 1* |
| False Negative | 81 | 75 | 6 | 2 |

*Specimen heavily spiked with M. avium

### Example 2

### Elisa Testing

The example is directed to comparative ability of a commercially available, USDA certified, Map ELISA test and a University of Florida College of Veterinary Medicine (UFCVM) Map ELISA test to diagnose Johne's disease in sera of cows with prior necropsy status confirmation.

Within the state of Florida, herds are screened using the Map Paracheck ELISA assay (Biocor, Omaha, Nebr.). A preliminary effort to assess comparative test sensitivity between the ELISA tests systems employed at the Florida′ State Diagnostic Laboratory at Live Oak and the UFCVM identified initial concerns, relative to the sensitivity of the respective tests. Forty sera had been independently tested using the Paracheck test at the state's Map diagnostic facility and then forwarded to UFCVM. The Paracheck ELISA data identified 6 of the 40 specimens tested as having significant ELISA titer: 1 inconclusive, 1 positive, and 5 strong positives. The UFCVM ELISA test results done on the same sera revealed 4 sera as being suspicious, 2 as positive, and 8 as strong-positive.

To assess the validity of the data reported from the respective institutions, necropsy files at the University of Florida College of Veterinary Medicine were reviewed in order to identify cows with well documented Johne's disease on gross and microscopic examination. The material available on each cow was then reviewed in order to identify the availability of feces and serum.

Study Population: The pathology reports from 2002-2005 were reviewed to identify dairy cows with necropsy confirmed Johne's disease for whom sera and fecal samples still existed. Nine animals meet the study entry criteria. In each case, an ELISA titer from the day of necropsy existed. The residual sera were divided into two aliquots, coded, and sent to the respective testing facilities. The previous UFCVM ELISA titers were used as a quality control check.

State of Florida Diagnostic Laboratory at Live Oak: The ParaCheck ELISA assays (Biocor, Omaha, Nebr.) were done in accordance with manufacturers' instruction and interpreted as prescribed by the kit insert. ELISA score of 0.00 to 0.49 is deemed negative; a score of 0.50 to 0.99 is deemed suspicious/inconclusive; and a score of 1.00 to 3.49 is deemed positive. A strong positive is any ELISA score of 3.50 or greater.

University of Florida College of Veterinary Medicine's Preabsorbed ELISA

Test: The in-house ELISA test was performed using a crude soluble protoplasmic antigen of M. avium (Allied Monitor, Missouri). Test sera were preabsorbed with Mycobacterium pheli. ELISA results were calculated from absorbance at OD 405 nm. All readings less than 1.6 optical density (OD) are deemed negative; readings between 1.6 and 1.99 were deemed suspicious/inconclusive. Readings of 2.0 to 2.5 were called positive. A strong positive was deemed any reading of above 2.5. All ELISA tests done at UFCVM were run in triplicate.

Results:

The comparative ELISA tests results are listed in table provided below. The Paracheck ELISA test identified one of the 9 Johne's disease cows. Another cow was deemed inconclusive. The in-house ELISA test correctly identified 6 of the nine animals. All three sera negative (range 0.49, 0.82, and 1.43) in UFCVM test were negative in the Paracheck test. Three cows (33%) with well documented Johne's disease were not identified by either ELISA test.

| | Paracheck | Paracheck | UF Map | UF |
|---|---|---|---|---|
| Cow # | Score | Interpretation | ELISA Score | Interpretation |
| 4371 | 0.00 | negative | 1.42 | negative |
| 3594 | 0.00 | negative | 0.49 | negative |
| 2894 | 0.00 | negative | 0.82 | negative |
| 3302 | 0.00 | negative | 2.13 | positive |
| 3036 | 0.06 | negative | 2.00 | positive |
| 3306 | 0.00 | negative | 2.00 | positive |
| 3147 | 0.34 | negative | 2.81 | strong positive |
| 205 | 0.87 | inconclusive | 2.53 | strong positive |
| 4496 | 5.44 | strong positive | 2.50 | positive |

### Example 3

### Quantitative PCR for Identification of Johne's Disease

Map Std 3 USDA kit 76 PAP1 10 pmol IS1311 (IS1&IS2)

| 1 | 2 | 3 | 4 | 5 | 6 | | | | 10 | 11 | 12 |
|---|---|---|---|---|---|---|---|---|---|---|---|
| Map Std | Map Std | Map Std | Map Std | Map Std | Map Std | | | | Pos | Pos | Pos |
| 10⁶ | 10⁵ | 10⁴ | 10⁶ | 10² | 10¹ | 7 | 8 | 9 | 0.33 ug/ul | 0.33 ug/ul | 0.33 ug/ul |
| 22.0 | 25.0 | 30.0 | 36.0 | 0.0 | 0.0 | | | | 19.4 | 18.9 | 18.2 |
| 76-1 | 76-1 | 76-2 | 76-2 | 76-3 | 76-3 | 76-4 | 76-4 | 76-6 | 76-6 | 76-7 | 76-7 |
| L | L | L | L | TNTC | TNTC | 0 | 0 | L | L | TNTC | TNTC |
| 30.1 | 27.5 | 30.3 | 28 | 32.1 | 29.5 | 0 | 0 | 35.7 | 33.6 | 29 | 26.7 |
| 76-8 | | 76-9 | 76-9 | 76-10 | 76-10 | 76-11 | 76-11 | 76-12 | 76-12 | 76-13 | 76-13 |
| 0 | 0 | + | + | M | M | fM | M | Mav | Mav | 0 | 0 |
| 0.0 | 0.0 | 30.4 | 30.9 | 35.7 | 34.2 | 29.1 | 27.1 | 23.5 | 22.6 | 0 | 0 |
| 76-14 | 76-14 | 76-15 | 76-15 | 76-16 | 76-16 | 76-17 | 76-17 | 76-18 | 76-18 | 76-19 | 76-19 |
| 0 | 0 | TNTC | TNTC | L | L | 0 | 0 | TNTC | TNTC | M | M |
| 0 | 0 | 30.7 | 29.3 | 29.2 | 29.2 | 0 | 0 | 33.2 | .30.9 | 38.5 | 35.4 |
| 76-20 | 76-20 | 76-21 | 76-21 | 76-22 | 76-22 | 76-23 | 76-23 | 76-24 | 76-24 | | |
| M | M | M | M | L | L | M | M | Mav | Mav | | |
| 31.6 | 30 | 33 | 31.6 | 31.1 | 28.6 | 36.2 | 0 | 24.9 | 23.8 | | |
| 76-25 | 76-25 | 76-26 | 76-26 | | | | | | | | |
| M | M | TNTC | TNTC | | | | | | | | |
| 29.1 | 28.1 | 30.2. | 28.6 | | | | | | | | |

The much referenced IS900 sequence (deemed specific to Map) provides diagnostic testing which identifies Mycobacterium avium subspecies paratuberculosis (Map). Another sequence, IS1311 offers the advantage of identifying both Mycobacterium avium subspecies paratuberculosis and Mycobacterium avium subspecies avium in one amplification, thereby reducing the time and expense of performing two separate test. The IS1311 sequence is basic to many mycobacteria. IS1/IS2 primers appear to identify pathogenic polymorphic mutation between Map and M. avium subspecies avium not detected by tests based upon the IS900 insertion sequence. The IS3/IS4 nested primers increase the sensitivity of the Map detection by primers based upon the IS1311 insertion sequence. The primers IS1/IS2 were therefore developed to meet our criteria of efficiency over culture analysis (seven hours vs. 42 days and extend the spectrum of organism identification. Standard direct PCR is not as efficient as real-time PCR. We have developed a labeled probe to function with our IS1/IS2 primers which enabled us to do real-time analysis which captures the stated diagnostic advantages stated above.

### Example 4

### Elisa Testing of Milk

This example identifies the correlation of Map DNA in milk based upon the J1J2 nested Map PCR technology and its correlation with its corresponding serum Map ELISA titer.

### Materials and Methods:

Study Population: Blood and milk samples were obtained from 81 Holstein dairy cows in a dairy research unit (DRU)'s Holstein herd.

### Sample Handling:

Raw Milk: Thirty-five to forty ml of milk was collected in a sterile 50 ml centrifuge tube from a randomly selected quarter by hand milking. Before collection, the teats were cleansed with alcohol. The first 10-15 ml of milk was discarded. The milk samples were centrifuged at 1000 g for 15 minutes and the supernatant discarded. The samples were washed three times using PBS (NaCL 43.3, Na2HPO4 11.4 g, KH2PO4 1.33 g, pH 7.3) and centrifuged at 500 g for 15 minutes. The pellet was re-suspended in 1 ml of PBS for cell counting, again centrifuged and re-suspended in 100 ul of 0.2 NaOH, boiled at 110 degrees Centigrade for 20 minutes to extract DNA, and centrifuged at 400 g for three minutes. Milk samples were collected over an approximately two and a half year period. For four cows, serial milk samples were collected over varying periods of time and analyzed using nested Map chain polymerase reaction test.
Blood Samples After cleansing with alcohol, 7-10 ml of blood was collected from the coccygeal vein into Vacutainer tubes (R) containing EDTA. Three ml of whole blood was added to 4 ml of Ficoll-Isopaque™ Plus Gradient (Amersham Pharmacia, density 1.078 g/ml) and centrifuged for 30 to 40 minutes at 400 g at 18 degrees Centigrade. The buffy layer was removed. The cells were then washed twice in PBS, and centrifuged at 500 g for 15 minutes. Cells were counted with a hemocytometer, re-suspended in 100 ul of 0.2 NaOH, boiled at 110 degrees Centigrade for 20 minutes to extract DNA, and centrifuged at 400 g for 3 minutes. Neutralization was not attempted
Agar Immunodiffusion Test (AGID): Petri dishes were poured with 1% agrose prepared in 0.1 M Tris-HCL buffer at pH 10. Well distances were 8 mm. Well sizes were 4 mm for the six peripheral wells and 3 mm for the central well. The peripheral well received 45 ul of the test serum. The central well was inoculated with 35 ul of a crude protoplasmic antigen (Allied Monitor, Missouri). Serum from a cow with documented Johne's disease constituted the positive control. Final analytical readings were done at 24 and 48 hours. The appearance of one or more clearly definable precipitation lines before or at 48 hours constituted a positive result. Absence of any precipitation lines constituted a negative result.
Preabsorbed ELISA Test: The ELISA tests were performed using a crude soluble protoplasmic antigen (Allied Monitor, Missouri). Test sera were Preabsorbed with Mycobacterium phlei. ELISA results were calculated from absorbance at OD 405 nm. All readings less than 1.6 optical density (OD) had been deemed negative; readings between 1.5 and 1.99 OD were deemed suspicious/inconclusive; and readings above 2.0 to 2.5 OD were called low positive. A high positive was deemed any reading 2.51 OD or above.
Map Nesting (Polymerase Chain Reaction (PCR): Samples were probed with primers P90P91 which recognized a 413 bp sequence of Mycobacterium avium subspecies paratuberculosis followed by a second set of primers J1J2 which overlapped and spanned a 333 base pair region within the insertion sequence. Primer exactness was checked using two sets of primers. Additional primer exactness was tested by submitting original samples to a set of P1P2 primers, recognizing a 427 bp sequence (IS1245) of Mycobacterium avium subspecies paratuberculosis (Map) and a third set of primers, DD2, DD3, probing for insertion sequence IS1311 which identifies a 180 bp sequence shared by Map. PCR products were sequenced (ICBR, University of Florida) for nucleotide homology using GenBank as the database. Homologies of 100% were obtained (Buergelt and Williams, 2004, Australian Vet. J. 82:497-503).

### Results:

Prevalence of Map in Milk Based on Single Specimen Analysis: Of the 81 dairy cows sampled with J1J2 nested PCR technology, 19 cows had Map DNA detected in the milk. The individual milk samples were compared with corresponding ELISA titers (Table 1). ELISA titers determined to be negative suspicious, positive and strong positive resulted in 4 (20%), 2 (15.4%), 2 (11.8%), and 9 (29%) milk samples being positive for Map DNA. The number of ELISA titers which tested negative for Map DNA in milk was 20, 13, 17 and 31, respectively. The best correlation between Map DNA in milk and corresponding serum ELISA titer on a single milk sample existed for samples with strong positive serum ELISA titers.
Observations of Map DNA in Milk Based upon Serial Specimens: Multiple milk samples were available on 81 dairy cows. In each case, Map was identified in two milk samples collected on separate dates. Four cows had greater than four specimens available for analysis (Tables 2, 3, and 4). Cow 3900 was monitored from July 2002 into November 2004. In those 45 months, Map was identified in its milk on four separate occasions.
Map Shedding From Individual Teats: Cow #6142 milk samples were obtained from its individual teats on six separate days (Table 4). While over all shedding was constant over 133 days, individual teats were negative on sampling. During the observation period, the ELISA titers varied between a high of 2.97 and 1.5.
Correlation between Map DNA in Milk and Necropsy Pathology: Nine dairy cows which had Map identified in one or more milk samples came to necropsy. Johne's disease was documented in all 9 cases.
Discussion: ELISA testing has been advocated as a voluntary herd management tool upon which individual producers could make decisions. An arbitrary absorbance value is thought to determine which animals are at greatest risk to the herd. The commercially licensed Map ELISA tests are used as herd management tools. Collins et al. have proposed that Map ELISA testing be used to remove the cows which are most infectious and not likely to survive another lactation (Collins, 2005, Clin. Diagn. Immunol. 12: 685-692). The underlying premise to this approach is that by removing the sickest animal, intra-herd dissemination of Map will be retarded. Fecal direct and nest polymerase chain reaction (PCR), fecal culture, and serological tests identify dairy cows which are infected with Map. Given the widespread prevalence of Map infection in large dairy herds and the potential from environmental re-introduction of Map into newly created dairy herds render total elimination of all infected animal as a short-term difficult goal. If selected emphasis is to be given to testing, a primary focus may be to eliminate those infected animal with sub-clinical disease which, in theory, constitute the greatest potential to introduce Map into the human food chain as well as enhance environmental contamination and intra-herd dissemination of Map. Cows with Map demonstrable in their milk constitute such animals.

From the data presented, a given ELISA titer has limited relevance as to whether or not a given cow is shedding Map into its milk. Based upon necropsy confirmation of established Johne's disease, the presence of Map antigen in milk appears to document prior spread of Map from the gastrointestinal tract. All nine cows for which subsequent necropsy reports became available demonstrated disseminated disease. Additionally, Map shedding into milk cannot be ascertained from a single milk sample. Map shedding can be irregular over an extended period of monitoring. A single negative nested Map PCR test does not rule out subsequent Map shedding into milk. To enhance a correct assessment as to the presence or absence of Map within milk from a given dairy cow requires multiple, individual milk sample, obtained at different dates being tested.

Another factor apparently affecting the presence or absence of Map in milk is the means by which a given sample is obtained. For a milk sample to be deemed adequate for analysis, the milk should be obtained from all four teats (pooled samples) and concentrated to increase the chances of detecting infected milk samples.

The observation of periods of Map shedding into milk, interspersed with periods of non-shedding, strongly suggests the importance of such factors as diet and/or environmental stress in governing a cow's ability to deal effectively with Map.

TABLE 1
Correlation of Serum Map ELISA Titers and Detection of
Map DNA in Individual Milk Samples

Nested PCR

| | | Number of | |
|---|---|---|---|
| ELISA Titer | Number of | Positive | |
| Serum | Negative Tests | Tests | Percentage |
| less than | 20 | 4 | 20% |
| 1.6 | | | |
| (negative) | | | |
| 1.6-1.99 | 13 | 2 | 15.4% |
| (suspicious) | | | |
| 2.0-2.5 | 17 | 2 | 11.8% |
| (positive) | | | |
| greater than | 31 | 9 | 29% |
| 2.51 | | | |
| (strong positive) | | | |

TABLE 2
Longitudinal Observations of Map DNA in Milk

| | | | Nesting Milk | |
|---|---|---|---|---|
| | Specimen Date | ELISA Titer | PCR | AGID |
| Cow #3900 | Jul. 23, 2002** | 3.1 | negative | negative |
| | Apr. 01, 2003** | 2.7 | negative | negative |
| | Apr. 28, 2003 | 6.1 | positive | negative |
| | Jun. 2, 2003 | 3.1 | negative | negative |
| | Jul. 1, 2003 | 3.7 | negative | negative |
| | Jul. 22, 2003 | 3.0 | negative | negative |
| | Feb. 17, 2004 | 2.76 | negative | negative |
| | Mar. 8, 2004** | 1.59 | positive | negative |
| | Mar. 22, 2004 | 2.85 | positive | negative |
| | Apr. 20, 2004 | 2.68 | negative | negative |
| | Jul. 1, 2004 | 3.55 | negative | negative |
| | Aug. 3, 2004** | 3.98 | negative | positive |
| | Aug. 25, 2004 | 5.54 | positive | positive |
| | Oct. 13, 2004 | 2.4 | negative | positive |

*Johne's disease documented at necropsy
**Map DNA identified within white blood cells by nested J1J2 PCR

TABLE 3
Serial Observations of Map DNA in Milk

| Cow | | Serum ELISA | Nested Milk | |
|---|---|---|---|---|
| Number | Date | Titer | PCR | AGID |
| #3763* | Sep. 10, 2003 | 1.8*** | positive | negative |
| | Sep. 12, 2003 | 1.5** | positive | negative |
| | Sep. 15, 2003 | 1.3** | positive | negative |
| | Sep. 16, 2003 | less than control | positive | negative |
| | Sep. 17, 2003 | 1.45** | negative | negative |
| | Sep. 18, 2003 | 1.66*** | positive | negative |
| #3485* | Sep. 25, 2003 | 1.68*** | negative | negative |
| | Sep. 26, 2003 | 1.85*** | positive | negative |
| | Sep. 29, 2003 | 1.5** | positive | negative |
| | Sep. 30, 2003 | 1.84*** | negative | negative |
| | Oct. 1, 2003 | 1.9*** | negative | negative |
| | Oct. 2, 2003 | 1.6*** | negative | negative |
| #3838* | Oct. 15, 2003 | 5.6**** | positive | positive |
| | Oct. 16, 2003 | 5.8**** | negative | positive |
| | Oct. 17, 2003 | 4.4**** | negative | positive |
| | Oct. 21, 2003 | 4.4**** | negative | positive |
| | Oct. 22, 2003 | 4.9**** | positive | positive |
| | Oct. 23, 2003 | 4.9**** | negative | positive |
| | Oct. 24, 2003 | 4.9**** | negative | positive |

Johne's disease confirmed at necropsy
**ELISA titer deemed negative (0-1.5)
***ELISA titer deemed suspicious (1.6-1.99)
****ELISA tier deemed strongly positive (greater than 2.51)

TABLE 4
Identification of Map DNA in Milk by Nested PCR From Individual
Teats Cow # 6142

| | |
|---|---|
| Nested PCR | ELISA |

| Date | RF | LF | LR | RR | Titer | AGID |
|---|---|---|---|---|---|---|
| Sep. 24, 2002 | + | + | − | + | 2.97**** | + |
| Dec. 10, 2002 | − | + | − | − | 1.5** | + |
| Dec. 30, 2002 | + | + | − | + | 2.0** | + |
| Jan. 21, 2003 | + | + | + | + | 2.68**** | − |
| Jan. 28, 2003 | + | + | + | + | 2.5***** | − |
| Feb. 4, 2003 | nt | − | − | − | 2.3***** | − |

RF = right front teat;
LF = left front teat;
LR = left rear teat;
RR = right rear teat
nt = not tested
+ = positive
− = negative
*Johne's disease documented at necropsy
**ELISA titer deemed negative (O-1.5)
***ELISA titer deemed suspicious (1.6-1.99)
****ELISA tier deemed strong positive (greater than 2.51)
*****ELISA titer deemed positive (2.0-2.5)

### Example 5

### Comparison of Two Direct Nested PCR Tests for the Detection of Mycobacterium avium Subspecies Paratuberculosis in Bovine Feces

Material and Methods:

Samples Analyzed: Four separate USDA Certification Kits, containing bovine fecal samples were analyzed. Kit number #1 (#F1-25 samples) was specifically created by USDA for the University of Florida College of Veterinary Medicine (UFCVM). Kits number #2 (#101-26 samples), and kit number #3 (#105-26 samples) were sent to a second UFCVM laboratory where they were tested for the presence of Map DNA by direct nested PCR. For the three sets of samples the investigators were blinded to the as to the code in each study.

DNA Extraction and PCR Procedure: All fecal extractions were done according to instructions from Mo Bio Laboratory Products Carlsbad, Calif.). Fecal samples were subjected to beating followed by a series of solutions for cell lysis, organic and inorganic precipitation. Binding of the DNA was achieved using a silica membrane with a high salt solution. DNA was then washed with an ethanol solution and eluded with an elution buffer. Samples were probed with two pairs P90-P91 with nested primers J1-J2 and IS1-IS2 with nested primers IS3-IS4, U.S. provisional patent No. 60/843,865.

Primers: Primers P90-P91 specifically recognize a 413 base pair sequence of Map IS900. Primers J1-J2 overlap and span a 333 base pair region within the insertion sequence. Primers IS1-IS2 recognize a 242 base pair sequence of Map IS1311 and primers IS3-IS4 overlap and span a 104 base pair region within the insertion sequence. Positive and negative controls were used in each of the reactions.

Statistical Analysis: Kappa coefficient was used as a measure of agreement between direct fecal nested Map PCR test results and kits keys provided by USDA. For this study, the test results provided by USDA were considered as “true” state of infection. The following categories were used for kappa test interpretation: poor agreement: less than 0.20; fair agreement: 0.21 to 0.40; moderate agreement: 0.41 to 0.60; good agreement: 0.61 to 0.80; very good agreement: 0.80 to 1.00. Fisher's Exact Test was used to test whether there was any non-random association between both variables of the two direct fecal nested Map PCR test results and provided culture results. This test was chosen because in all the cases the tables were highly imbalanced (low values in the cell for both variables). The right-sided probability value was used considering the alternative hypothesis of a positive association between both results (observations tending to lie in upper and lower right cells of the 2×2 contingency table). Data were analyzed using SAS statistical package for Windows (Version 9.00) using the PROC FREQ procedure. Values of P less than 0.05 were considered significant for all tests.

In the analysis, sensitivity and specificity of direct fecal nested Map PCR tests were estimated as a gold standard, the kit key for each specimen as negative as negative or to positive to infection. Kappa coefficient, sensitivity and specificity were estimated using Win Episcope 2.0 software (Win Episcope 2.0). Ninety-five percent confidence intervals (CI) were constructed for all estimates.

Results:

Estimation of sensitivity and specificity and kappa coefficients for the samples from kits 1 to 3 for the two direct fecal nested Map PCR test results with keys provided by keys provided by USDA are presented in Table 5.

Fisher's Exact Test used to test the null hypothesis of no association between nested PCR tests (J1-J2 and IS3-IS4) and origin laboratory key in p-values less than 0.0001 for both cases. The data indicates that in two cases there was sufficient evidence to reject the null hypothesis (i.e., there was significant statistical association between results of nested PCR tests and origin laboratory key). The agreement between FecaMap test results and fecal culture provided by USDA was good for both sets of primers.

Table 6 defines the comparison sensitivity data presented in terms of false positive and false negative results observed in the three USDA laboratory certification kits. Table 7 reports of the approximate time requirement for removal of the PCR inhibitors in feces and PCR testing achieve using the FecaMap™ system in doing ten fecal specimens.

Discussion:

The U.S. herd management response to combat Johne's disease has been to advocate a voluntary policy of selective herd testing. The commercially licensed Map ELISA test are not diagnostic tests, but rather herd management tests. In theory, their function is to contribute to overall herd welfare. Additionally, it has been proposed that Map ELISA testing be used to remove the cows which are most infectious and not likely to survive another lactation. The underlying premise to this approach is that by removing the sickest animal, over time, natural selection would take over.

The ELISA tests currently available lack acceptable sensitivity. In an evaluation of five antibody detection tests for the diagnosis of bovine paratuberculosis using serum samples from 359 dairy cattle in seven paratuberculosis-free herds and 2,094 cattle in seven Map-infected dairy herds, it was determined that the antibody tests lacked acceptable sensitivity (Collins et al., 2005, Clin. Diagn. Immunol. 12:685-692). Both the ParaCheck (Biocor, Omaha, Nebr.) and HerdCheck (IDEXX Laboratories Inc. Westbrook, Me.) ELISA tests done in accordance with manufacturers' instruction and interpreted as prescribed by the kit insert, identified less than 29% of fecal culture positive cows. Diagnostic specificity among the five ELISA tests evaluate ranged from 84.7% to 86.5%. Linear regression analysis of quantitative results showed a low correlation co-efficiency. A more positive relationship could be shown between the number of mycobacterium in feces and ELISA positivity. With low number of Map in their feces, a mean of 13.3% of infected cows were ELISA positive. At progressively higher fecal culture scores, the mean percentage of positive antibody assays were 27.3% 54.9% and 78.4% respectively.

A variable affecting statistical analysis was the inclusion in the USDA fecal samples of a specimen heavily spiked with M. avium which was detected by both nested J1-J2 and IS3-IS4 but not P90-P91. Comparable results were achieved with another set of base and nested IS900 primers under development in our laboratory. Despite the inclusion of the M. avium spiked sample, the P90-P91/J1-J2 combination's sensitivity were 89.3% and 90.5% where as those of the IS1-IS2/IS3-IS4 combination were 96.4% and 95.2% respectively with a fecal culture vs. PCR Kappa value of 0.903.

A potential drawback to the utilization of the nested J1-J2, or IS3-IS4) which are based upon the IS 900 and IS1311 sequence respectively is that all nested primers tested in-house to date have identified M. avium, when the organism is spiked into diagnostic fecal specimens.

The FecaMap™ sets of primers are based on the IS1311 sequence Nevertheless, the IS1-IS2 primers show a demonstrable superiority in comparison to the IS900 P90-P91 pairing. The IS1311 primer pairs identify only 6-8 copies where as primers based upon the IS900 sequence identify 14-18 copies. A case can be argued USDA's insistence on near absolute specificity for Map has resulted in development of specificity for Map refined done at the expense of sensitivity.

The current study indicates that selective strains of Map may have genetic constituency not adequately identified by IS900 sequence based primers. Herman-Taylor's theory that the incorporation of foreign DNA into M. avium lead to ultimate evolution of the Map phenotype appears to have a real foundation based upon the comparison of the base primers studied. Primers which identified both Map and M. avium identified more positive USDA fecal samples than did those based foreign DNA encompassed in the IS 900 sequence into background M. avium species led to the development of current Map phenotype. The M. avium based test produced a statistically significant rate of correspondence in sera from necropsy documented cows (67%). The Paracheck Map ELISA test identified only 11% of the diseased animals.

With respect to limitation of Map intra-herd dissemination or protection of a nation's food supply, there is no effective herd management schema in place. The development of direct fecal nested PCR tests and possibly the EVELISA test provide a potential foundation for development of herd management schema which are applicable to these two, under-addressed issues. The direct fecal nested Map PCR test process, FecaMap™, produces results in seven hours or less. With conversion of the test to automation, the time required can be significantly reduced.

TABLE 5
Statistical comparison of P90-P91/J1-J2 versus IS1-IS2/IS3-IS4
primers on three USDA laboratory certification kit fecal specimens

| | P90-P91/J1-J2 | IS1-IS2/IS3-IS4 |
|---|---|---|
| Sensitivity | 89.3% | 96.5% |
| Specificity | 90.5% | 95.2% |
| Kappa Coefficient | 0.753 | 0.903 |
| Interpretation | Good agreement | Very good agreement |

TABLE 6
Comparison of primers P90-P91, IS1-IS2, P90-P91/J1-J2
and IS1-IS2/IS3-IS4 false positive and false negative observed
on three USDA certification kit fecal specimens
Primers

| | P90-P91 | IS1-IS2 | P90-P91/J1-J2 | IS1-IS2/IS3-IS4 |
|---|---|---|---|---|
| False Positive | 20 | 26 | 2 | 1* |
| False Negative | 81 | 75 | 6 | 2 |

*Specimen heavily spiked with M. avium

TABLE 7
Time requirement for removal of fecal PCR inhibitors and PCR testing
(n = 10).

| Procedure | Time Required |
|---|---|
| 1. Decontamination | 90 minutes |
| 2. PCR | 120 minutes |
| 3. Nested PCR* | 120 minutes |
| 4. Gel for nesting | See above |
| Approximate Total Time To Results | 7 hours |

*(including concomitant gel preparation)

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A composition of matter comprising:
(a) a PCR primer set specific for Mycobacterium avium subsp. paratuberculosis (MAP) comprising the primers identified in any one of the following primer sets:

| | |
|---|---|
| Primer | |
| Set | SEQ ID NOs: |
| | | |
|---|---|---|
| 1 | 1 | 2 |
| 2 | 3 | 4 |
| 3 | 8 | 9 |
| 4 | 11 | 12 |
| 5 | 14 | 15 |
| 6 | 17 | 18 |
| 7 | 20 | 21 |
| 8 | 23 | 24 |
| 9 | 26 | 27 |
| 10 | 29 | 30 |
| 11 | 32 | 33 |
| 12 | 35 | 36 |
| 13 | 38 | 39 |
| 14 | 41 | 42 |
| 15 | 44 | 45 |
| 16 | 47 | 48 |
| 17 | 50 | 51 |
| 18 | 53 | 54 |
| 19 | 56 | 57 |
| 20 | 59 | 60 |
| 21 | 62 | 63 |
| 22 | 65 | 66 |
| 23 | 68 | 69 |
| 24 | 71 | 72 |
| 25 | 74 | 75 |
| 26 | 77 | 78 |
| 27 | 80 | 81 |
| 28 | 83 | 84 |
| 29 | 86 | 87 |
| 30 | 89 | 90 |
| 31 | 92 | 93 |
| 32 | 95 | 96 |
| 33 | 98 | 99 |
| 34 | 101 | 102 |
| 35 | 104 | 105 |
| 36 | 107 | 108 |
| 37 | 110 | 111 |
| 38 | 113 | 114 |
| 39 | 116 | 117 |
| 40 | 119 | 120 |
| 41 | 122 | 123 |
| 42 | 125 | 126 |
| 43 | 128 | 129 |
| 44 | 131 | 132 |
| 45 | 134 | 135 |
| 46 | 137 | 138 |
| 47 | 140 | 141 |
| 48 | 143 | 144 |
| 49 | 146 | 147 |
| 50 | 149 | 150 |
| 51 | 152 | 153 |
| 52 | 155 | 156 |
| 53 | 158 | 159 |
| 54 | 161 | 162 |
| 55 | 164 | 165 |
| 56 | 167 | 168 |
| 57 | 170 | 171 |
| 58 | 173 | 174 |
| 59 | 176 | 177 |
| 60 | 179 | 180 |
| 61 | 182 | 183 |
| 62 | 185 | 186 |
| 63 | 188 | 189 |
| 64 | 191 | 192 |
| 65 | 194 | 195 |
| 66 | 197 | 198 |
| 67 | 200 | 201 |
| 68 | 203 | 204 |
| 69 | 205 | 206 |
| 70 | 207 | 208 |
| 71 | 209 | 210 |
| 72 | 211 | 212 |

(b) a PCR primer set specific for Mycobacterium avium subsp. paratuberculosis (MAP), wherein each respective primer set also comprises a probe polynucleotide:

| Primer | |
|---|---|
| Set | SEQ ID NO: |
| 73 | 8, 9 and 10 |
| 74 | 11, 12 and 13 |
| 75 | 14, 15 and 16 |
| 76 | 17, 18 and 19 |
| 77 | 20, 21 and 22 |
| 78 | 23, 24 and 25 |
| 79 | 26, 27 and 28 |
| 80 | 29, 30 and 31 |
| 81 | 32, 33 and 34 |
| 82 | 35, 36 and 37 |
| 83 | 38, 39 and 40 |
| 84 | 41, 42 and 43 |
| 85 | 44, 45 and 46 |
| 86 | 47, 48 and 49 |
| 87 | 50, 51 and 52 |
| 88 | 53, 54 and 55 |
| 89 | 56, 57 and 58 |
| 90 | 59, 60 and 61 |
| 91 | 62, 63 and 64 |
| 92 | 65, 66 and 67 |
| 93 | 68, 69 and 70 |
| 94 | 71, 72 and 73 |
| 95 | 74, 75 and 76 |
| 96 | 77, 78 and 79 |
| 97 | 80, 81 and 82 |
| 98 | 83, 84 and 85 |
| 99 | 86, 87 and 88 |
| 100 | 89, 90 and 91 |
| 101 | 92, 93 and 94 |
| 102 | 95, 96 and 97 |
| 103 | 98, 99 and 100 |
| 104 | 101, 102 and 103 |
| 105 | 104, 105 and 106 |
| 106 | 107, 108 and 109 |
| 107 | 110, 111 and 112 |
| 108 | 113, 114 and 115 |
| 109 | 116, 117 and 118 |
| 110 | 119, 120 and 121 |
| 111 | 122, 123 and 124 |
| 112 | 125, 126 and 127 |
| 113 | 128, 129 and 130 |
| 114 | 131, 132 and 133 |
| 115 | 134, 135 and 136 |
| 116 | 137, 138 and 139 |
| 117 | 140, 141 and 142 |
| 118 | 143, 144 and 145 |
| 119 | 146, 147 and 148 |
| 120 | 149, 150 and 151 |
| 121 | 152, 153 and 154 |
| 122 | 155, 156 and 157 |
| 123 | 158, 159 and 160 |
| 124 | 161, 162 and 163 |
| 125 | 164, 165 and 166 |
| 126 | 167, 168 and 169 |
| 127 | 170, 171 and 172 |
| 128 | 173, 174 and 175 |
| 129 | 176, 177 and 178 |
| 130 | 179, 180 and 181 |
| 131 | 182, 183 and 184 |
| 132 | 185, 186 and 187 |
| 133 | 188, 189 and 190 |
| 134 | 191, 192 and 193 |
| 135 | 194, 195 and 196 |
| 136 | 197, 198 and 199 |
| 137 | 200, 201 and 202 |

(c) a PCR primer set specific for Mycobacterium avium subsp. paratuberculosis (MAP) comprising the following combinations of primers:
Combinations of Primers (SEQ ID NOs:)

| | | |
|---|---|---|
| 8 and 9 and | 38 and 39 | ; |
| | 41 and 42 | ; |
| | 44 and 45 | ; |
| | 47 and 48 | ; or |
| | 50 and 51 | |
| 11 and 12 and | 53 and 54 | ; |
| | 56 and 57 | ; |
| | 59 and 60 | ; |
| | 62 and 63 | ; or |
| | 65 and 66 | |
| 14 and 15 and | 68 and 69 | ; |
| | 71 and 72 | ; |
| | 74 and 75 | ; |
| | 77 and 78 | ; or |
| | 80 and 81 | |
| 17 and 18 and | 83 and 84 | ; |
| | 86 and 87 | ; |
| | 89 and 90 | ; |
| | 92 and 93 | ; or |
| | 95 and 96 | |
| 20 and 21 and | 98 and 99 | ; |
| | 101 and 102 | ; |
| | 104 and 105 | ; |
| | 107 and 108 | ; or |
| | 110 and 111 | |
| 23 and 24 and | 113 and 114 | ; |
| | 116 and 117 | ; |
| | 119 and 120 | ; |
| | 122 and 123 | ; or |
| | 125 and 126 | |
| 26 and 27 and | 128 and 129 | ; |
| | 131 and 132 | ; |
| | 134 and 135 | ; |
| | 137 and 138 | ; or |
| | 140 and 141 | |
| 29 and 30 and | 143 and 144 | ; |
| | 146 and 147 | ; |
| | 149 and 150 | ; |
| | 152 and 153 | ; or |
| | 155 and 156 | |
| 32 and 33 and | 158 and 159 | ; |
| | 161 and 162 | ; |
| | 164 and 165 | ; |
| | 167 and 168 | ; or |
| | 170 and 171 | |
| 35 and 36 and | 173 and 174 | ; |
| | 176 and 177 | ; |
| | 179 and 180 | ; |
| | 182 and 183 | ; or |
| | 185 and 186 | |
| 188 and 189 and | 203 and 204 | ; |
| | 205 and 206 | ; |
| | 207 and 208 | ; |
| | 209 and 210 | ; or |
| | 211 and 212 | |
| 191 and 192 and | 203 and 204 | ; |
| | 205 and 206 | ; |
| | 207 and 208 | ; |
| | 209 and 210 | ; or |
| | 211 and 212 | |
| 194 and 195 and | 203 and 204 | ; |
| | 205 and 206 | ; |
| | 207 and 208 | ; |
| | 209 and 210 | ; or |
| | 211 and 212 | |
| 197 and 198 and | 203 and 204 | ; |
| | 205 and 206 | ; |
| | 207 and 208 | ; |
| | 209 and 210 | ; or |
| | 211 and 212 | ; or |
| 200 and 201 and | 203 and 204 | ; |
| | 205 and 206 | ; |
| | 207 and 208 | ; |
| | 209 and 210 | ; or |
| | 211 and 212 | |

(d) a PCR primer set specific for Mycobacterium avium subsp. paratuberculosis (MAP) comprising the following combinations of primers:
Combinations of Primers (SEQ ID NOs:)

| | | |
|---|---|---|
| 8 and 9 and 10 and | 38 and 39 | ; |
| | 41 and 42 | ; |
| | 44 and 45 | ; |
| | 47 and 48 | ; or |
| | 50 and 51 | |
| 11 and 12 and 13 | 53 and 54 | ; |
| and | 56 and 57 | ; |
| | 59 and 60 | ; |
| | 62 and 63 | ; or |
| | 65 and 66 | |
| 14 and 15 and 16 | 68 and 69 | ; |
| and | 71 and 72 | ; |
| | 74 and 75 | ; |
| | 77 and 78 | ; or |
| | 80 and 81 | |
| 17 and 18 and 19 | 83 and 84 | ; |
| | 86 and 87 | ; |
| | 89 and 90 | ; |
| | 92 and 93 | ; or |
| | 95 and 96 | |
| 20 and 21 and 22 | 98 and 99 | ; |
| and | 101 and 102 | ; |
| | 104 and 105 | ; |
| | 107 and 108 | ; or |
| | 110 and 111 | |
| 23 and 24 and 25 | 113 and 114 | ; |
| and | 116 and 117 | ; |
| | 119 and 120 | ; |
| | 122 and 123 | ; or |
| | 125 and 126 | |
| 26 and 27 and 28 | 128 and 129 | ; |
| and | 131 and 132 | ; |
| | 134 and 135 | ; |
| | 137 and 138 | ; or |
| | 140 and 141 | |
| 29 and 30 and 31 | 143 and 144 | ; |
| and | 146 and 147 | ; |
| | 149 and 150 | ; |
| | 152 and 153 | ; or |
| | 155 and 156 | |
| 32 and 33 and 34 | 158 and 159 | ; |
| and | 161 and 162 | ; |
| | 164 and 165 | ; |
| | 167 and 168 | ; or |
| | 170 and 171 | |
| 35 and 36 and | 173 and 174 | ; |
| | 176 and 177 | ; |
| | 179 and 180 | ; |
| | 182 and 183 | ; or |
| | 185 and 186 | |
| 188 and 189 and | 203 and 204 | ; |
| 190 and | 205 and 206 | ; |
| | 207 and 208 | ; |
| | 209 and 210 | ; or |
| | 211 and 212 | |
| 191 and 192 and | 203 and 204 | ; |
| 193 and | 205 and 206 | ; |
| | 207 and 208 | ; |
| | 209 and 210 | ; or |
| | 211 and 212 | |
| 194 and 195 and | 203 and 204 | ; |
| 196 and | 205 and 206 | ; |
| | 207 and 208 | ; |
| | 209 and 210 | ; or |
| | 211 and 212 | |
| 197 and 198 and | 203 and 204 | ; |
| 199 and | 205 and 206 | ; |
| | 207 and 208 | ; |
| | 209 and 210 | ; or |
| | 211 and 212 | ; or |
| 200 and 201 and | 203 and 204 | ; |
| 202 and | 205 and 206 | ; |
| | 207 and 208 | ; |
| | 209 and 210 | ; or |
| | 211 and 212 | |

(e) a PCR primer set specific for Mycobacterium avium subsp. paratuberculosis (MAP) comprising the following combinations of primers:
Combinations of Primers
(SEQ ID NOs:)

| | | |
|---|---|---|
| 8 and 9 and | 38 and 39 and 40 | ; or |
| | 41 and 42 and 43 | ; or |
| | 44 and 45 and 46 | ; or |
| | 47 and 48 and 49 | ; or |
| | 50 and 51 and 52 | |
| 11 and 12 | 53 and 54 and 55 | ; or |
| and | 56 and 57 and 58 | ; or |
| | 59 and 60 and 61 | ; or |
| | 62 and 63 and 64 | ; or |
| | 65 and 66 and 67 | |
| 14 and 15 | 68 and 69 and 70 | ; or |
| and | 71 and 72 and 73 | ; or |
| | 74 and 75 and 76 | ; or |
| | 77 and 78 and 79 | ; or |
| | 80 and 81 and 82 | |
| 17 and 18 | 83 and 84 and 85 | ; or |
| and | 86 and 87 and 88 | ; or |
| | 89 and 90 and 91 | ; or |
| | 92 and 93 and 94 | ; or |
| | 95 and 96 and 97 | |
| 20 and 21 | 98 and 99 and 100 | ; or |
| and | 101 and 102 and 103 | ; or |
| | 104 and 105 and 106 | ; or |
| | 107 and 108 and 109 | ; or |
| | 110 and 111 and 112 | |
| 23 and 24 | 113 and 114 and 115 | ; or |
| and | 116 and 117 and 118 | ; or |
| | 119 and 120 and 121 | ; or |
| | 122 and 123 and 124 | ; or |
| | 125 and 126 and 127 | |
| 26 and 27 | 128 and 129 and 130 | ; or |
| and | 131 and 132 and 133 | ; or |
| | 134 and 135 and 136 | ; or |
| | 137 and 138 and 139 | ; or |
| | 140 and 141 and 142 | |
| 29 and 30 | 143 and 144 and 145 | ; or |
| and | 146 and 147 and 148 | ; or |
| | 149 and 150 and 151 | ; or |
| | 152 and 153 and 154 | ; or |
| | 155 and 156 and 157 | |
| 32 and 33 | 158 and 159 and 160 | ; or |
| and | 161 and 162 and 163 | ; or |
| | 164 and 165 and 166 | ; or |
| | 167 and 168 and 169 | ; or |
| | 170 and 171 and 172 | |
| 35 and 36 | 173 and 174 and 175 | ; or |
| and | 176 and 177 and 178 | ; or |
| | 179 and 180 and 181 | ; or |
| | 182 and 183 and 184 | ; or |
| | 185 and 186 and 187 | |

(f) a PCR primer set specific for Mycobacterium avium subsp. paratuberculosis (MAP) comprising the following combinations of primers:
Combinations of Primers
(SEQ ID NOs:)

| | | |
|---|---|---|
| 8 and 9 and | 38 and 39 and 40 | ; or |
| 10 and | 41 and 42 and 43 | ; or |
| | 44 and 45 and 46 | ; or |
| | 47 and 48 and 49 | ; or |
| | 50 and 51 and 52 | |
| 11 and 12 | 53 and 54 and 55 | ; or |
| and 13 and | 56 and 57 and 58 | ; or |
| | 59 and 60 and 61 | ; or |
| | 62 and 63 and 64 | ; or |
| | 65 and 66 and 67 | |
| 14 and 15 | 68 and 69 and 70 | ; or |
| and 16 and | 71 and 72 and 73 | ; or |
| | 74 and 75 and 76 | ; or |
| | 77 and 78 and 79 | ; or |
| | 80 and 81 and 82 | |
| 17 and 18 | 83 and 84 and 85 | ; or |
| and 19 and | 86 and 87 and 88 | ; or |
| | 89 and 90 and 91 | ; or |
| | 92 and 93 and 94 | ; or |
| | 95 and 96 and 97 | |
| 20 and 21 | 98 and 99 and 100 | ; or |
| and 22 and | 101 and 102 and 103 | ; or |
| | 104 and 105 and 106 | ; or |
| | 107 and 108 and 109 | ; or |
| | 110 and 111 and 112 | |
| 23 and 24 | 113 and 114 and 115 | ; or |
| and 25 and | 116 and 117 and 118 | ; or |
| | 119 and 120 and 121 | ; or |
| | 122 and 123 and 124 | ; or |
| | 125 and 126 and 127 | |
| 26 and 27 | 128 and 129 and 130 | ; or |
| and 28 and | 131 and 132 and 133 | ; or |
| | 134 and 135 and 136 | ; or |
| | 137 and 138 and 139 | ; or |
| | 140 and 141 and 142 | |
| 29 and 30 | 143 and 144 and 145 | ; or |
| and 31 and | 146 and 147 and 148 | ; or |
| | 149 and 150 and 151 | ; or |
| | 152 and 153 and 154 | ; or |
| | 155 and 156 and 157 | |
| 32 and 33 | 158 and 159 and 160 | ; or |
| and 34 and | 161 and 162 and 163 | ; or |
| | 164 and 165 and 166 | ; or |
| | 167 and 168 and 169 | ; or |
| | 170 and 171 and 172 | |
| 35 and 36 | 173 and 174 and 175 | ; or |
| and 37 and | 176 and 177 and 178 | ; or |
| | 179 and 180 and 181 | ; or |
| | 182 and 183 and 184 | ; or |
| | 185 and 186 and 187 | |

(g) an isolated polynucleotide comprising any one of SEQ ID NOs: 1 through 212 or an isolated polynucleotide comprising at least 8 consecutive nucleotides of any one of SEQ ID NOs: 1 through 212;
(h) an isolated polynucleotide comprising at least 8 consecutive nucleotides of any one of SEQ ID NOs: 1 through 212, wherein said polynucleotide has a maximum length that is equal to the number of nucleotides associated with said specific SEQ ID NO:;
(i) an isolated polynucleotide that is fully complementary to:
(1) any one of SEQ ID NO: 1 through 212;
(2) a polynucleotide comprising at least 8 consecutive nucleotides of any one of SEQ ID NOs: 1 through 212; or
(3) a polynucleotide comprising at least 8 consecutive nucleotides of any one of SEQ ID NOs: 1 through 212, wherein said polynucleotide has a maximum length that is equal to the number of nucleotides associated with said specific SEQ ID NO:; or
(j) an isolated polynucleotide comprising a contiguous/consecutive span of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 consecutive nucleotides of SEQ ID NO: 5, 7 or 213 provided that said contiguous/consecutive span of nucleotides includes at least 8 consecutive nucleotides of a primer or probe selected from any one of SEQ ID NOs: 1-4 and 8-212 or polynucleotides fully complementary to any one of SEQ ID NOs: 1-4 and 8-212.

2. The primer set or isolated polynucleotide according to claim 1, wherein one or more of said primers is labeled or said polynucleotide is labeled.

3. The primer set or isolated polynucleotide according to claim 2, wherein said label is a fluorescent label.

4. The primer set or isolated polynucleotide according to claim 2, wherein said label is a radioisotope.

5. The primer set or isolated polynucleotide according to claim 2, wherein said label is biotin.

6. A method of detecting the presence of Mycobacterium avium subsp. paratuberculosis (MAP) in a sample from individual suspected of being infected with MAP, said method comprising the steps of:
(a) providing a sample from the individual suspected of being infected with MAP;
(b) treating the sample to solubilize the nucleic acids therein;
(c) forming a PCR reaction solution comprising:
(i) at least a portion of the solubilized nucleic acids from step (b);
(ii) any one of the PCR primer sets according to claim 1;
(iii) a mixture of nucleoside triphosphate monomers; and
(iv) a PCR polymerase in a buffered solution;
(d) carrying out a polymerase chain reaction on the PCR reaction solution to amplify any MAP-specific nucleic acid which is specific for the particular primer set used to a level sufficient for detection; and
(e) detecting the presence of amplified MAP-specific nucleic acid in the resulting solution which is specific for the particular primer set used; wherein the detection of the amplified MAP-specific nucleic acid which is specific for the particular primer set used indicates that MAP is present in the individual.

7. The method according to claim 6 wherein the sample is a fecal sample from an individual.

8. The method according to claim 7, wherein said individual is a bovine.

9. The method according to claim 6, wherein the primer set comprises primer set 2.

10. The method according to claim 9, wherein the primer set further comprises SEQ ID NO: 1.

11. The method according to claim 9, wherein the primer set further comprises SEQ ID NO: 2.

12. The method according to claim 9, wherein the primer set further comprises SEQ ID NO: 1 and SEQ ID NO: 2.

13. The method according to claim 6, wherein the primer set comprises a polynucleotide comprising at least 8 contiguous nucleotides of SEQ ID NO: 3 and a polynucleotide comprising at least 8 contiguous nucleotides of SEQ ID NO: 4.

14. The method according to claim 13, wherein the primer set further comprises a polynucleotide comprising at least 8 contiguous nucleotides of SEQ ID NO: 1.

15. The method according to claim 13, wherein the primer set further comprises a polynucleotide comprising at least 8 contiguous nucleotides of SEQ ID NO: 2.

16. The method according to claim 13, wherein the primer set further comprises a polynucleotide comprising at least 8 contiguous nucleotides of SEQ ID NO: 1 and a polynucleotide comprising at least 8 contiguous nucleotides of SEQ ID NO: 2.

17. The method according to claim 6, wherein the detection of the presence of amplified MAP-specific nucleic acid comprises gel electrophoresis of the amplified MAP-specific nucleic acid solution and staining of the resulting gel to visualize the band of the MAP-specific nucleic acid specific for the particular primer set used.

18. The method according to claim 17, wherein at least one of the oligonucleotides in the primer set or at least one of the nucleoside triphosphate monomers contains a label which will be incorporated into the amplified MAP-specific nucleic acid and can be used for the detection of the amplified MAP-specific nucleic acid.

19. A method of detecting the presence of MAP in a sample from individual suspected of being infected with MAP using a nested PCR procedure, said method comprising the steps of:
(a) providing a sample from the individual suspected of being infected with MAP;
(b) treating the sample to solubilize the nucleic acids therein;
(c) forming a first PCR reaction solution containing at least a portion of the solubilized nucleic acids from step (b), a first PCR primer set, a first mixture of nucleoside triphosphate monomers, and a first PCR polymerase in a first buffered solution, wherein the first primer set comprises a first pair of oligonucleotides as set forth in primer set 1, 3, 4, 5, 6, 7, 8, 9, 10, 11, 12, 63, 64, 65, 66 or 67 or fragments of said first pair of oligonucleotides that are at least 8 consecutive nucleotides in length;
(d) performing a first polymerase chain reaction on the first PCR reaction solution to amplify any MAP-specific nucleic acid which is specific for the first primer set used;
(e) forming a second PCR reaction solution containing at least a portion of the PCR-reacted first PCR reaction solution from step (d), a second PCR primer set, a second mixture of nucleoside triphosphate monomers, and a second PCR polymerase in a second buffered solution, wherein the second primer set comprises a second pair of oligonucleotides as set forth in primer set 2, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 68, 69, 70, 71 or 72 or fragments of said second pair of oligonucleotides that are at least 8 consecutive nucleotides in length;
(f) performing a second polymerase chain reaction on the second PCR reaction solution to amplify any MAP-specific nucleic acid which is specific for the second primer set used to a level sufficient for detection; and
(g) detecting the presence of amplified MAP-specific nucleic acid in the resulting solution from step (f) which specific for the second primer set; wherein the detection of the amplified MAP-specific nucleic acid which is specific for the second primer set indicates that MAP is present in the individual.

20-26. (canceled)

27. A method of identifying animals having Johne's disease comprising:
a) obtaining sera from an animal suspected of having Johne's disease;
b) contacting a crude soluble protoplasmic antigen of M. avium with sera from said animal (test sera) and a control sera; and
c) detecting the binding of antibodies to said crude protoplasmic antigen, wherein an animal having Johne's disease is identified when the amount of test sera antibody bound to the crude soluble antigen is greater than the amount of a control sera antibody bound to said crude soluble antigen.

28. The method according to claim 27, wherein the sera obtained from said animal has been preabsorbed with Mycobacterium phei.

29. The method according to claim 27, wherein said detecting comprises contacting the antibodies of said test sera and said control sera with a labeled antibody.

30. The method according to claim 29, wherein said antibody is labeled with a fluorophore, an enzyme, or a radiolabel.

31. The method according to claim 6, further comprising the detection of amplified gene product with a probe.

32. The method according to claim 31, wherein said probe comprises a label that is a fluorescent dye or radiolabel.

33. The method according to claim 32, wherein said probe comprises a fluorescent dye and a quencher.

34. The method according to claim 33, wherein said probe is 5′-/56-FAM/CAC ACT GTC GAC GAT CGC/31ABlkFQ/-3′.
